(19) United States
(12) Patent Application Publication
(10) Pub. No.: US 2016/0186073 A1
(43) Pub. Date: Jun. 30, 2016

(54) METHODS AND SYSTEMS FOR PROCESSING CELLULOSIC BIOMASS

(71) Applicant: SHELL OIL COMPANY, Houston, TX (US)

(72) Inventors: Joseph Broun POWELL, Houston, TX (US); Glenn Charles KOMPLIN, Katy, TX (US); Juben Nemchand CHHEDA, Houston, TX (US)

(21) Appl. No.: 14/980,262

(22) Filed: Dec. 28, 2015

Related U.S. Application Data
(60) Provisional application No. 62/097,744, filed on Dec. 30, 2014.

(51) Int. Cl. C10G 3/00; C07C 29/88; C07C 29/86; C07C 29/80; B01J 19/24; C07C 45/00; C07C 45/82; C07C 45/85; C07C 45/80; C07C 29/00; C07D 307/33

(52) U.S. Cl. CPC C10G 3/49; C07C 29/00; C07C 29/88; C07C 29/86; C07C 29/80; C07D 307/33; C07C 45/00; C07C 45/82; C07C 45/85; C07C 45/80; B01J 19/24; B01J 19/245; B01J 2219/24

(57) ABSTRACT

Separation of a product of digestion of cellulosic biomass solids may be challenging due to the various components contained therein. Methods and systems for processing cellulosic biomass, particularly a reaction product of a hydrothermal reaction containing lignin-derived products, such as phenolics, and organic salts, comprise providing acid solution to the organic salt containing process stream to convert the organic acid salts to acids to for further processing.

### CROSS-REFERENCE TO RELATED APPLICATION

The present application claims the benefit of pending U.S. Provisional Application Ser. No. 62/097,744, filed 30 Dec., 2014, the entire disclosure of which is hereby incorporated by reference.

### BACKGROUND OF THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

The present disclosure generally relates to processing of cellulosic biomass solids, and, more specifically, to methods and systems for processing a reaction product comprising lignin that may be obtained by a hydrothermal reaction of cellulosic biomass.

A number of substances of commercial significance may be produced from natural sources, including biomass. Cellulosic biomass may be particularly advantageous in this regard due to the versatility of the abundant carbohydrates found therein in various forms. As used herein, the term “cellulosic biomass” refers to a living or formerly living biological material that contains cellulose. The lignocellulosic material found in the cell walls of higher plants is the world's largest source of carbohydrates. Materials commonly produced from cellulosic biomass may include, for example, paper and pulpwood via partial digestion, biofuels, including bioethanol by fermentation.

Development of fossil fuel alternatives derived from renewable resources have received recent attention. Cellulosic biomass has garnered particular attention in this regard due to its abundance and the versatility of the various constituents found therein, particularly cellulose and other carbohydrates. Despite promise and intense interest, the development and implementation of bio-based fuel technology has been slow. Existing technologies have heretofore produced fuels having a low energy density (e.g., bioethanol) and/or that are not fully compatible with existing engine designs and transportation infrastructure (e.g., methanol, biodiesel, Fischer-Tropsch diesel, hydrogen, and methane). Moreover, conventional bio-based processes have typically produced intermediates in dilute aqueous solutions (>50% water by weight) that are difficult to further process. Energy- and cost-efficient processes for processing cellulosic biomass into fuel blends having similar compositions to fossil fuels would be highly desirable to address the foregoing issues and others.

Further, in addition to the desired carbohydrates, other substances may be present within cellulosic biomass that can be especially problematic to deal with in an energy- and cost-efficient manner. For example, during cellulosic biomass processing, significant quantities of lignin present in cellulosic biomass may lead to fouling of processing equipment, potentially leading to costly system down time. The lignin can also lead to realization of a relatively low conversion of the cellulosic biomass into useable substances per unit weight of feedstock.

As evidenced by the foregoing, an efficient conversion of cellulosic biomass into fuel blends and other materials is a complex problem that presents immense engineering challenges. The present disclosure addresses these challenges and provides related advantages as well.

### SUMMARY OF THE INVENTION

Recovery of various compounds from a product of reactions involving cellulosic biomass may be challenging due to the various components contained therein, including lignin and salts from the biomass. The present disclosure provides methods and systems for processing cellulosic biomass, where the insoluble organic salts in the process are converted to soluble acids that can be further processed to valuable products.

In some embodiments, liquid-liquid extraction (or solvent extraction) is provided to isolate the phases so that the phases can be further processed independent of each other or liquid-liquid phase separation is provided to separate the phases. As used herein, liquid-liquid extraction refers to extraction of a substance from one liquid phase (e.g., an aqueous phase) into a different liquid phase (e.g., a non-aqueous phase) based on the relative solubility of the substance in each of the phases. Accordingly, liquid-liquid extraction can provide for a certain amount of extraction of various compounds from the aqueous phase. These compounds can include certain light compounds can be further processed into a fuels product and can include those with a normal boiling point of less than about 150 degrees C., including but not limited to monohydric alcohols (e.g., mono-oxygenates), aldehydes, ketones, acids, and any combination thereof. Liquid-liquid extraction solvent can be provided from various process streams and product streams in the process.

Once separated, the aqueous phase can be subject to flashing with reduced steam stripping effect because it contains a reduced amount of non-water miscible compounds compared to the reaction product with multiple liquid phases because a major portion of the non-water miscible compounds, such as phenols and various oils, are in the non-aqueous phase(s). If light compounds are still present in sufficient quantity in the aqueous phase after the liquid-liquid extraction or separation, flashing of the aqueous phase can recover the light compounds. To improve product separation, the non-aqueous phase(s) can be subject to a viscosity reducing reaction, including one or more lignin depolymerization reactions.

Distillation of the non-aqueous phase(s) with reduced water can achieve adequate product separation at a lower temperature where higher temperatures would be needed to achieve similar efficient product separations in distillation of the reaction product without water removal or one that has been subject to flashing prior to distillation. Optionally, the separated non-aqueous phase(s) can be subject to a flashing step prior to distillation to recover an overhead fraction comprising light compounds and a bottom fraction if desired. The remaining portion of the bottom fraction of the non-aqueous phase(s) can be provided to distillation.

The product fractions from distillation of the non-aqueous phase(s) can include at least an overhead fraction comprising light compounds, a middle fraction comprising lignin and lignin-derived products, including phenols, and a bottom fraction comprising heavy compounds with normal boiling points above about 350° C. The distillation can be performed to provide more than one middle fraction. For instance, distillation can be operated to provide a first middle fraction with a normal boiling point of about 150 to 250 degrees C. and a second middle fraction with a higher normal boiling point of 200 to 350 degrees C. Distillation can also be operated to provide a middle fraction with a normal boiling point of about 150 to 350 degrees C.

The light compounds recovered from the aqueous phase and the non-aqueous phase(s) can optionally be combined for further processing into a higher hydrocarbon products that may be useful for fuels application. The higher hydrocarbons can be optionally distilled to different fractions for optimal liquid-liquid extraction separation. Such fraction may be rich in aromatic content (for example, aromatics-rich hydrocarbon fraction) or lower in aromatic content (lower aromatics hydrocarbon fraction) or other higher hydrocarbon fraction from distilling the higher hydrocarbon products (e.g., products from condensation reaction). Optionally, to improve product separation, the non-aqueous phase(s) can be subject to a viscosity reducing reaction, including one or more lignin depolymerization reactions.

In some embodiment, at least a portion of the lignin in the stream is precipitated by providing at least a portion of the higher hydrocarbon product. The precipitated lignin can be carried to the subsequent distillation unit as a slurry then separated in the distillation unit or can be at least in part be separated in the lignin precipitation unit.

Such higher hydrocarbons can be provided to the at least partially depolymerized lignin stream to precipitate at least a portion of the lignin and optionally may also be provided to the liquid-liquid extraction for use as part of the extraction solvent. The higher hydrocarbons may be optionally distilled to different fractions for optimal liquid-liquid extraction separation. Such fraction may be rich in aromatic content (for example, aromatics-rich hydrocarbon fraction) or lower in aromatic content (lower aromatics hydrocarbon fraction) or other higher hydrocarbon fraction from distilling the higher hydrocarbon products (e.g., products from condensation reaction).

In particular, the current disclosure provides a method comprising: heating cellulosic biomass solids, molecular hydrogen, a catalyst capable of activating molecular hydrogen, and a digestion solvent in a reactor in a first reaction zone to produce a first reaction product; providing an acid solution to the first reaction product to produce an acidified first reaction product; providing at least a portion of the acidified first reaction product to a separation zone comprising a liquid-liquid phase separation unit or a liquid-liquid extraction unit to recover an aqueous stream and a non-aqueous stream, wherein the aqueous stream comprises a major portion of water in said portion of the first reaction product; providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and providing at least a portion of the non-aqueous stream to a distillation unit to obtain at least an overhead fraction, a middle fraction, and a bottom fraction.

In addition, the current disclosure provides another method comprising: heating cellulosic biomass solids, molecular hydrogen, a catalyst capable of activating molecular hydrogen, and a digestion solvent in a reactor in a first reaction zone to produce a first reaction product; heating at least a portion of the first reaction product, molecular hydrogen, and a catalyst capable of activating molecular hydrogen in a reactor in a second reaction zone to produce a second reaction product; providing an acid solution to the second reaction product to produce an acidified second reaction product; providing at least a portion of the acidified second reaction product to a separation zone comprising a liquid-liquid phase separation unit or a liquid-liquid extraction unit to recover an aqueous stream and a non-aqueous stream, wherein the aqueous stream comprises a major portion of water in said portion of the second reaction product; providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and providing at least a portion of the non-aqueous stream to a distillation unit to obtain at least an overhead fraction, a middle fraction, and a bottom fraction.

The acid solution may be provided before or after an optional gas separator unit where a vapor fraction comprising compounds with a normal boiling point of 100 degrees C. or lower prior to providing the acid solution is recovered from at least one of the first reaction product or the second reaction product.

In addition, the current disclosure provides another method comprising: heating cellulosic biomass solids, molecular hydrogen, a catalyst capable of activating molecular hydrogen, and a digestion solvent in a reactor in a first reaction zone to produce a first reaction product; providing at least a portion of the first reaction product to a separation zone comprising a liquid-liquid phase separation unit or a liquid-liquid extraction unit to recover an aqueous stream and a non-aqueous stream, wherein the aqueous stream comprises a major portion of water in said portion of the first reaction product, providing an acid solution to the aqueous stream prior to providing the aqueous stream to an aqueous stream separation zone; providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and providing at least a portion of the non-aqueous stream to a distillation unit to obtain at least an overhead fraction, a middle fraction, and a bottom fraction.

In addition, the current disclosure provides another method comprising: heating cellulosic biomass solids, molecular hydrogen, a catalyst capable of activating molecular hydrogen, and a digestion solvent in a reactor in a first reaction zone to produce a first reaction product; heating at least a portion of the first reaction product, molecular hydrogen, and a catalyst capable of activating molecular hydrogen in a reactor in a second reaction zone to produce a second reaction product; providing at least a portion of the second reaction product to a separation zone comprising a liquid-liquid phase separation unit or a liquid-liquid extraction unit to recover an aqueous stream and a non-aqueous stream, wherein the aqueous stream comprises a major portion of water in said portion of the second reaction product; providing an acid solution to the aqueous stream prior to providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and providing at least a portion of the non-aqueous stream to a distillation unit to obtain at least an overhead fraction, a middle fraction, and a bottom fraction.

In addition, the current disclosure provides a system comprising: a first reaction zone comprising a reactor configured heat cellulosic biomass solids, molecular hydrogen, a catalyst capable of activating molecular hydrogen, and a digestion solvent to form a first reaction product; and a separation zone comprising: an inlet to receive an acid solution in fluid communication with an outlet of the reactor in the first reaction zone and with the liquid-liquid phase separation unit or a liquid-liquid extraction unit; a liquid-liquid phase separation unit or a liquid-liquid extraction unit having an inlet in fluid communication with an outlet of the reactor in the first reaction zone to receive the first reaction product, wherein the liquid-liquid phase separation unit or the liquid-liquid extraction unit is configured to provide an aqueous stream and a non-aqueous stream, wherein the aqueous phase comprises a major portion of water in said portion of the first reaction product; an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction.

In addition, the current disclosure provides a system comprising: a first reaction zone comprising a reactor configured heat cellulosic biomass solids, molecular hydrogen, a catalyst capable of activating molecular hydrogen, and a digestion solvent to form a first reaction product; a second reaction zone comprising a reactor in fluid communication with an outlet of the reactor in the first reaction zone to receive the first reaction product, wherein the reactor in the second reaction zone is configured to heat a second reaction content comprising the first reaction product, molecular hydrogen, and a catalyst capable of activating molecular hydrogen to form a second reaction product; and a separation zone comprising: an inlet to receive an acid solution in fluid communication with an outlet of the reactor in the second reaction zone and with the liquid-liquid phase separation unit or the liquid-liquid extraction unit; a liquid-liquid phase separation unit or a liquid-liquid extraction unit having an inlet in fluid communication with an outlet of the reactor in the second reaction zone to receive second first reaction product, wherein the liquid-liquid phase separation unit or the liquid-liquid extraction unit is configured to provide an aqueous stream and a non-aqueous stream, wherein the aqueous stream comprises a major portion of water in said portion of the first reaction product; an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction; wherein the distillation unit is in fluid communication with an inlet of the reactor in the first reaction zone to provide at least a portion of the middle fraction.

Yet in another embodiment, the processing zone may further comprise a second distillation unit having an inlet in fluid communication with an outlet of the further processing unit to receive at least a portion of the higher hydrocarbon products from the further processing unit, wherein the distillation unit is configured to provide at least an aromatics rich hydrocarbon fraction, optionally lower aromatics hydrocarbon fraction and a heavy fraction; wherein the second distillation unit is in fluid communication with an inlet of the lignin precipitation unit to provide at least a portion of the aromatics rich fraction or a lower aromatics hydrocarbon fraction.

The features and advantages of embodiments provided by the present disclosure will be readily apparent to one having ordinary skill in the art upon a reading of the description of the embodiments that follows.

### BRIEF DESCRIPTION OF THE DRAWINGS

The following figures are included to illustrate certain aspects of the present disclosure, and should not be viewed as an exclusive embodiment. The subject matter disclosed is capable of considerable modifications, alterations, combinations, and equivalents in form and function, as will occur to one having ordinary skill in the art and the benefit of this disclosure.

FIG. 1 shows a schematic of an illustrative embodiment for processing cellulosic biomass according to some aspects provided by this disclosure.

FIG. 2 shows a schematic of another illustrative embodiment for processing cellulosic biomass according to some aspects provided by this disclosure.

FIG. 3 shows an illustrative embodiment of an aqueous separation zone involved in processing of cellulosic biomass according to some aspects provided by this disclosure.

### DETAILED DESCRIPTION OF THE INVENTION

The present disclosure generally provides methods for processing cellulosic biomass into a fuel product, particularly processing of a reaction product obtained from hydrothermal digestion of cellulosic biomass solids. Cellulosic biomass is particularly advantageous because of the versatility of the abundant carbohydrates found therein in various forms. As used herein, the term “cellulosic biomass” refers to a living or formerly living biological material that contains cellulose. The lignocellulosic material found in the cell walls of higher plants is one of the world's largest sources of carbohydrates.

Unless otherwise specified, it is to be understood that use of the terms “biomass” or “cellulosic biomass” may be synonymous. The cellulosic biomass may be in any size, shape, or form. The cellulosic biomass may be natively present in any of these solid sizes, shapes, or forms, or they may be further processed prior to digestion. The cellulosic biomass may be chopped, ground, shredded, pulverized, and the like to produce a desired size prior to hydrothermal digestion. The cellulosic biomass may be washed (e.g., with water, an acid, a base, combinations thereof, and the like) prior to digestion taking place.

Any type of suitable cellulosic biomass source may be used. Suitable cellulosic biomass sources may include, for example, forestry residues, agricultural residues, herbaceous material, municipal solid wastes, waste and recycled paper, pulp and paper mill residues, and any combination thereof. Thus, in some embodiments, a suitable cellulosic biomass may include, for example, corn stover, straw, bagasse, miscanthus, sorghum residue, switch grass, bamboo, water hyacinth, hardwood, hardwood chips, hardwood pulp, softwood, softwood chips, softwood pulp, duckweed and any combination thereof. Leaves, roots, seeds, stalks, husks, and the like may be used as a source of the cellulosic biomass. Common sources of cellulosic biomass may include, for example, agricultural wastes (e.g., corn stalks, straw, seed hulls, sugarcane leavings, nut shells, and the like), wood materials (e.g., wood or bark, sawdust, timber slash, mill scrap, and the like), municipal waste (e.g., waste paper, yard clippings or debris, and the like), and energy crops (e.g., poplars, willows, switch grass, alfalfa, prairie bluestream, corn, soybeans, and the like). The cellulosic biomass may be chosen based upon considerations such as, for example, cellulose and/or hemicellulose content, lignin content, growing time/season, growing location/transportation cost, growing costs, harvesting costs, and the like.

Plants have primary cell walls and secondary cell walls. The primary cell wall contains three major polysaccharides (cellulose, pectin, and hemicellulose) and one group of glycoproteins. The secondary cell wall also contains polysaccharides polymeric lignin that is covalently crosslinked to hemicellulose. The complex mixture of constituents that is co-present with the cellulose can make its processing difficult, as discussed hereinafter. Lignin, in particular, may be an especially difficult constituent to deal with.

When converting cellulosic biomass into fuel blends and other materials, cellulose and other complex carbohydrates therein can be extracted and transformed into simpler organic molecules, which can be further processed thereafter to higher hydrocarbons. The higher hydrocarbons produced are useful in forming transportation fuels, such as synthetic gasoline, diesel fuel, and jet fuel, as well as industrial chemicals. As used herein, the term “higher hydrocarbons” refers to hydrocarbons having an oxygen to carbon ratio less than the oxygen to carbon ratio of at least one component of the biomass feedstock. The higher hydrocarbon predominantly contains C4 to C30 hydrocarbons, more preferably C6 to C18 hydrocarbons. Higher hydrocarbons may contain aromatics and alkanes. As used herein the term “hydrocarbon” refers to an organic compound comprising primarily hydrogen and carbon atoms, which is also an unsubstituted hydrocarbon. In certain embodiments, the hydrocarbons of the invention also comprise heteroatoms (i.e., oxygen sulfur, phosphorus, or nitrogen) and thus the term “hydrocarbon” may also include substituted hydrocarbons.

Digestion is one way in which cellulose and other complex carbohydrates may be converted into a more usable form. Digestion processes can break down cellulose and other complex carbohydrates within cellulosic biomass into simpler, soluble carbohydrates that are suitable for further transformation through downstream further processing reactions. As used herein, the term “soluble carbohydrates” refers to monosaccharides or polysaccharides that become solubilized in a digestion process. Illustrative carbohydrates that may be present in cellulosic biomass solids include, for example, sugars, sugar alcohols, celluloses, lignocelluloses, hemicelluloses, and any combination thereof.

The issues associated with converting cellulosic biomass into fuel blends (biofuels) in an energy- and cost-efficient manner using digestion are not only complex, but they are entirely different than those that are encountered in the digestion processes commonly used in the paper and pulpwood industry. Since the intent of cellulosic biomass digestion in the paper and pulpwood industry is to retain a solid material (e.g., wood pulp), incomplete digestion is usually performed at low temperatures (e.g., less than about 200° C.) for a fairly short period of time (e.g., between two to four hours). In contrast, digestion processes suitable for converting cellulosic biomass into fuel blends and other materials are ideally configured to maximize yields by solubilizing as much of the original cellulosic biomass charge as possible in a high-throughput manner. Paper and pulpwood digestion processes also typically remove lignin from the raw cellulosic biomass prior to pulp formation. Although digestion processes used in connection with forming fuel blends and other materials may likewise remove lignin prior to digestion, these extra process steps may affect the energy efficiency and cost of the biomass conversion process. The presence of lignin during high-conversion cellulosic biomass digestion may be particularly problematic.

Production of soluble carbohydrates for use in fuel blends and other materials via routine modification of paper and pulpwood digestion processes is not believed to be economically feasible for a number of reasons. Simply running the digestion processes of the paper and pulpwood industry for a longer period of time to produce more soluble carbohydrates is undesirable from a throughput standpoint. Use of digestion promoters such as strong alkalis, strong acids, or sulfites to accelerate the digestion rate can increase process costs and complexity due to post-processing separation steps and the possible need to protect downstream components from these agents. Accelerating the digestion rate by increasing the digestion temperature can actually reduce yields of biofuels due to thermal degradation of soluble carbohydrates that can occur at elevated digestion temperatures, particularly over extended periods of time. Once produced by digestion, soluble carbohydrates are very reactive and can rapidly degrade to produce caramelans and other heavy ends degradation products or byproducts, especially under higher temperature conditions, such as above about 150 degrees C. Use of higher digestion temperatures can also be undesirable from an energy efficiency standpoint. Any of these difficulties can defeat the economic viability of fuel blends derived from cellulosic biomass.

A particularly effective manner in which soluble carbohydrates may be formed is through hydrothermal digestion, and in which the soluble carbohydrates may be converted into more stable compounds is through subjecting them to one or more catalytic reductions, which may include hydrogenation and/or hydrogenolysis reactions. Stabilizing soluble carbohydrates through conducting one or more catalytic reduction reactions may allow digestion of cellulosic biomass to take place at higher temperatures than would otherwise be possible without unduly sacrificing yields. Depending on the reaction conditions and catalyst used, reaction products formed because of conducting one or more catalytic reduction reactions on soluble carbohydrates may comprise one or more alcohol functional groups, particularly including triols, glycol, monohydric alcohols, and any combination thereof, some of which may also include residual carbonyl functionality (e.g., an aldehyde or a ketone). The compounds in the alcoholic component can be described as oxygenates where the compounds comprise one or more oxygen-containing functional groups, such as a hydroxyl group or a carbonyl group. Non-limiting examples of oxygenates include an aldehyde, a ketone, an alcohol, furan, an ether, or any combination thereof. Such reaction products are more thermally stable than soluble carbohydrates and may be readily transformable into fuel blends and other materials through conducting one or more downstream further processing reactions. In addition, the foregoing types of reaction products are good solvents in which a hydrothermal digestion may be performed, thereby promoting solubilization of soluble carbohydrates as their reaction products. However, such reaction products also form carboxylates salts that are lost for further processing as products and are eliminated as salts. Examples of such carboxylate salts may include any of C1-C6 carboxylic acids salts such as formates, acetates, lactates, propionate, levulinates salts.

Hydrothermal digestion of a cellulosic biomass can include heating of the cellulosic biomass and a digestion solvent in the presence of molecular hydrogen and a catalyst capable of activating the molecular hydrogen (which can also be referred to herein as a “hydrogen-activating catalyst” or “hydrocatalytic catalyst”). In such approaches, the hydrothermal digestion of cellulosic biomass and the catalytic reduction of soluble carbohydrates produced therefrom may take place in the same vessel, which can be referred to as “in situ catalytic reduction reaction processes.” As such, digestion processes suitable for converting cellulosic biomass into fuel blends and other materials are preferably configured to maximize yields by solubilizing as much of the original cellulosic biomass charge as possible in a high-throughput manner. In situ catalytic reduction reaction processes may also be particularly advantageous from an energy efficiency standpoint, since hydrothermal digestion of cellulosic biomass is an endothermic process, whereas catalytic reduction reactions are exothermic. Thus, the excess heat generated by the in situ catalytic reduction reaction(s) may be utilized to drive the hydrothermal digestion with little opportunity for heat transfer loss to occur, thereby lowering the amount of additional heat energy input needed to conduct the digestion.

Typically, hydrothermal reactions of cellulosic biomass, including hydrothermal digestion and catalytic reduction, generates a reaction product that contains a variety of components, ranging from lighter compounds, such as an alcoholic component, to heavier compounds, including lignin and lignin-derived compounds, such as phenolics, and various compounds in between. The reaction product can also exist in more than one phase, depending on certain conditions, such as temperature and pressure. Because the reaction product is a mixture of compounds, further processing certain compounds to desired products can be challenging. For instance, further processing of certain light compounds in the reaction product to desired fuel products can be challenging, at least from an economics point of view, because other middle-boiling compounds, such as lignin and lignin-derived compounds also present, can limit further processing options of the components in the reaction product.

For example, the lignin present in the reaction product may lead to fouling of processing equipment, potentially leading to costly system down time. The reaction product is a mixture of various compounds and typically in more than one liquid phase. One phase of the reaction product is usually water. While feasible, distillation of a reaction product containing water into various product fractions typically requires higher temperatures and pressures compared to one with reduced water. The extra energy needed for the higher temperatures and pressures can translate into additional costs. While the water in the reaction product can be removed through flashing prior to distillation into product fractions, doing so can result in steam stripping where water vapors carry some phenols into the overhead fraction. Removal of phenols during flashing can result in less efficient product separation in a subsequent distillation process.

The problem of loss of yield from salt formation is address by reverting at least a portion of the carboxylate salts to form free organic acids by adding inorganic acids to the process streams at certain locations thus recovering carboxylic acids and forming inorganic salt and thereby prevent loss of yield. The acid solution may contain sulfuric acids, nitric acids, phosphoric acids and hydrochloric acids. Optionally, acid solution from the wash unit from washing the biomass can be used in this process particularly since any contaminants in the acid wash solution will bypass the catalysts capable of activating hydrogen. Such acid solutions and wash methods and systems are described in detail in co-pending US patent Application Publication Nos. US20150166681, US20150165488, US20150166682, US20150167238, each disclosure is herein incorporated by reference in its entirety. Preferably, the inorganic acid is added to the process stream so the process stream is acidified to a pH of 3 or less.

In an embodiment, liquid-liquid phase separation may be provided to isolate the phases so that the phases can be further processed independent of each other. The reaction product can comprise an aqueous phase and one or more non-aqueous phases, which can be mixed with one another in an emulsion. Two streams that result from the liquid-liquid phase separation process include a non-aqueous stream that contains a major portion of the non-aqueous phase(s) and an aqueous stream that contains a major portion of the aqueous phase. Alternatively, an extraction solvent can be providing to a liquid-liquid extraction to isolate the phases so that the phases can be further processed independent of each other. The reaction product can comprise an aqueous phase and one or more non-aqueous phases, which can be mixed with one another in an emulsion. In liquid-liquid extraction, an extraction solvent is added to the reaction product extract various compounds from the aqueous phase. Two streams that result from the liquid-liquid extraction process include: (i) a non-aqueous stream that contains a major portion of the non-aqueous phase(s) and a major portion of the extraction solvent, which contains the extracted compounds, and (ii) an aqueous stream that contains a major portion of the aqueous phase. Accordingly, the aqueous stream comprises a major portion of water present in the reaction product, while the non-aqueous stream comprises a minor portion of water present in the reaction product. The term “major” or grammatical equivalents thereof refers to greater than 50 wt %, including 55 wt % or greater, 60 wt % or greater, 70 wt % or greater, 75 wt % or greater, 80 wt % or greater, 85 wt % or greater, 90 wt % or greater, and 95 wt % or greater. The term “minor” or grammatical equivalents thereof means up to 50 wt %, including an amount in a range of 0.1 wt % and up to 50 wt %, up to 45 wt %, up to 40 wt %, up to 35 wt %, up to 30 wt %, up to 25 wt %, up to 20 wt %, up to 15 wt %, up to 10 wt %, or up to 5 wt %.

Once separated, the aqueous stream can be subject to flashing with reduced steam stripping effect because it contains a reduced amount of non-water miscible compounds compared to the reaction product with multiple liquid phases because a major portion of the non-water miscible compounds, such as phenols, are in the non-aqueous stream. If light compounds are still present in sufficient quantity in the aqueous stream after the liquid-liquid extraction, flashing of the aqueous stream can recover the light compounds. As mentioned, light compounds can include those with a normal boiling point of less than about 150 degrees C., including but not limited to monohydric alcohols, aldehydes, ketones, acids, and any combination thereof. The terms “light,” “middle-boiling,” and “heavy,” or grammatical variations thereof, refer to various compounds relative to one another based on their normal boiling points.

If extraction solvent is used, selection of solvent, operating conditions, and the mechanism of contacting the solvent and reaction product permit control over the level or degree of compound extraction. In particular, the amount of extraction solvent added to the reaction product correlates to the amount of compounds extracted into the solvent phase from the aqueous phase. For instance, in a mixture of reaction product and extraction solvent that contains a relatively small amount of solvent, such as about 1 part solvent phase per 9 parts of aqueous phase, the extraction of compounds into the solvent is small as compared to when a larger amount of solvent is contacted with the aqueous phase, such as a mixture with about 9 parts solvent per one part of aqueous phase. That is, the degree of extraction increases with increasing amounts of extraction solvent used in the liquid-liquid extraction process. As such, it is possible to configure the liquid-liquid extraction process to extract a major portion of compounds from the aqueous phase to a degree where further handling of the aqueous phase for additional product recovery, such as flashing to recover light compounds, may not be needed. The addition of an extraction solvent can also facilitate coalescing of the phases of the reaction product by assisting with the breaking of the emulsion.

If an extraction solvent is used, at least a portion of the extraction solvent can come from a higher hydrocarbon product from further processing at least a portion of the vapor fraction, at least a portion of the non-aqueous stream, and/or at least a portion of the distillate overhead fraction to provide a higher hydrocarbon product in the further processing zone. At least a portion of the higher hydrocarbon product may be provided to the liquid-liquid extraction unit, wherein the extraction solvent comprises said portion of the higher hydrocarbon product, in addition to its use in precipitation of lignin. In another embodiment at least a portion of the extraction solvent can come from at least a portion of the middle fraction from the distillation unit. In yet another embodiment, at least a portion of the non-aqueous stream may be flashed to recover an overhead fraction comprising light compounds and a bottom fraction comprising alkyl cyclohexanol, which a first portion may be provided to the liquid-liquid extraction unit. The second portion of the bottom fraction from flashing can be provided to the distillation unit. The overhead fraction from the flashing contains a major portion of compounds with a normal boiling point of less than 150 degrees C. in such portion of the non-aqueous stream, and the bottom fraction from flashing contains compounds with a normal boiling point of 150 degrees C. or higher.

Prior to distillation, optionally a portion of the lignin may be removed by at least precipitating a portion of the lignin by providing at least a portion of the higher hydrocarbon product. The precipitated lignin can be carried to the subsequent distillation unit as a slurry then separated in the distillation unit or can be at least in part be separated in the lignin precipitation unit. The lignin can be separated by filtration, centrifugation, hydroclones, settling tanks (or gravity settlers), dual flasher (where more than one flasher is present so as to burn off lignin in of the flasher while the one of the other flasher is in use to separate the precipitated lignin) and the likes. One such lignin separation method is described in detail in a co-pending U.S. application Ser. No. 14/485,869, which disclosure is herein incorporated by reference in its entirety. It has been found that the lignin in the stream at least in part precipitates upon addition of higher hydrocarbon products, particularly in the presence of an aromatics-rich hydrocarbon stream, thus facilitating the removal of lignin from the stream to lessen equipment fouling.

The product fractions from the non-aqueous stream can include at least an overhead fraction comprising light compounds, a middle fraction comprising lignin and lignin-derived products, including cyclic alcohols and phenols, and a bottom fraction comprising waste. The non-aqueous stream can also comprise an amount of longer diols and mono-oxygenates (such as those with greater than C4) formed from carbohydrates, which can be recovered in the middle fraction. The distillation can be performed to provide more than one middle fractions. For instance, distillation can be operated to provide a first middle fraction with a normal boiling point of about 150 to 250 degrees C. and a second middle fraction with a higher normal boiling point of 200 to 350 degrees C. Distillation can also be operated to provide a middle fraction with a normal boiling point of about 150 to 350 degrees C.

As known to one of ordinary skill in the art, the overlap in the range of normal boiling point of the middle fractions is at least due to the distribution of compounds during the distillation process. For instance, trace amounts of light compounds may be found in a middle fraction and/or a bottom fraction, and vice versa, trace amounts of heavy compounds may be found in the overhead fraction and/or middle fraction. Likewise, there would be an overlap of compounds in the more than one middle fractions, if present, which leads to an overlap of normal boiling point of the middle fractions.

For the first distillation, in general, the first middle fraction may be referred to as the phenols fraction because it tends to capture compounds with normal boiling points around that of phenolic compounds. It is understood that the first middle fraction can contain other compounds from the second middle fraction as well as those with normal boiling points falling outside of about 150 to 250 degrees C. as discussed above. Similarly, the second middle fraction may be referred to as the lignin oligomers fraction because it tends to capture heavier compounds with normal boiling points around that of lignin oligomers. It is understood that the second middle fraction can contain other compounds from the first middle fraction as well as those with normal boiling points falling outside of about 200 to 350 degrees C. as discussed above. The middle fraction, whether or not it is in one fraction or more than one fraction, contains a major portion of the phenols in the reaction product fed to the distillation process.

The light compounds recovered from the aqueous stream and the non-aqueous stream can optionally be combined for further processing into a higher hydrocarbons or fuels product. A middle fraction recovered from the non-aqueous stream can be recycled in the process. The middle fraction may further comprise lignin oligomers. Optionally, to improve product separation of the non-aqueous stream, the non-aqueous stream can be subject to a viscosity reducing reaction, including one or more lignin depolymerization reactions. Accordingly, the methods and systems described in the present disclosure can provide for improved yields and lower costs, including reducing or minimizing the need to employ expensive high pressure distillation and separations to process the reaction product.

The term “distillation” has its ordinary meaning, which refers to a method of recovering volatile compounds based on their normal boiling points. Distillation can be done in one stage or multiple stages. A one-stage of single stage distillation may be referred to as “flashing” or a grammatical equivalent, including “flash” or “flasher.” Flashing produces an overhead fraction and a bottom fraction and typically does not produce a middle product fraction. Multi-stage distillation produces various product fractions, including an overhead fraction and a bottom fraction, as well as one or more middle fractions. The manner in which flashing or multi-stage distillation can be carried out is known to one of ordinary skill in the art.

Unless otherwise specified, it is understood that the mention of various components, such as compounds, products, phases fractions, portions, streams, etc., in this disclosure refers to “at least a portion of” such component, whether or not the phrase “at least a portion of” is included therewith. The terms “hydrocarbon compounds,” in the second reaction product refer to compounds comprising hydrogen and carbon atoms that do not have a phenolic functional group, which is a hydroxyl group (—OH) bonded to an aromatic hydrocarbon group. Illustrative, non-limiting hydrocarbon compounds include alkanes, alkenes, cycloalkanes and their alkyl substituents or derivatives, and cycloalkenes and their alkyl substituents or derivatives, which can be suitable for use in fuel composition, for instance gasoline or diesel. For instance, illustrative hydrocarbon compounds can include but are not limited to any one of cyclohexane, cyclohexene, propyl cyclopentane, propyl cyclopentene, propyl cyclohexane, propyl cyclohexene, anisole, propyl benzene, methyl propyl benzene, and oxygenated hydrocarbons such as cyclohexanone or methyl cyclohexanone.

Methods and systems described in this disclosure can provide for lignin reversion, e.g., reversion of lignin to phenols and conversion of phenolics derived from lignin to hydrocarbons. As mentioned, processing of cellulosic biomass can include hydrothermally digesting cellulosic biomass and carrying out a catalytic reduction reaction of soluble carbohydrates, including in one reactor. This can be achieved via in situ catalytic reduction reaction, which involves heating the cellulosic biomass and a digestion solvent in the presence of molecular hydrogen and a catalyst capable of activating molecular hydrogen. The hydrothermal digestion and catalytic reduction can generate a first reaction product comprising phenolics derived from lignin in the cellulosic biomass and an alcoholic component formed from a catalytic reduction reaction of soluble carbohydrates derived from the cellulosic biomass.

The term “alcoholic component” refers to an oxygenate where the oxygenate can be a monohydric alcohol, a glycol or diol, a triol, or any combination thereof. As used herein, the terms “glycol” or diol will refer to compounds containing two alcohol functional groups, two alcohol functional groups and a carbonyl functionality, or any combination thereof. It is understood that the alcoholic component also includes carbonyls that have carbonyl functionality. As used herein, the term “carbonyl functionality” will refer to an aldehyde functionality or a ketone functionality. In some embodiments, a glycol may comprise a significant fraction of the reaction product. Although a glycol may comprise a significant fraction of the reaction product, it is to be recognized that other alcohols, including triols and monohydric alcohols, for example, may also be present, as well as other oxygenates such as the ketone equivalent of the alcohol. Further, any of these alcohols may further include a carbonyl functionality.

As used herein, the term “triol” will refer to compounds containing three alcohol functional groups, three alcohol functional groups and a carbonyl functionality, and any combination thereof. As used herein, the term “monohydric alcohol” will refer to compounds containing one alcohol functional group, one alcohol functional group and a carbonyl functionality, and any combination thereof. The term “phenolics” or “phenols” has its ordinary meaning, which generally refers to a class of compounds that contain a hydroxyl group (—OH) bonded to an aromatic hydrocarbon group. The term “lignin oligomers” refers to a class of compounds with more than one lignin monomer, two or more aromatic rings, and a molecular weight of up to 300,000 Daltons. It is understood that lignin is a complex, amorphous, three-dimensional polymer having a structure based on phenylpropane. The molecular structure of lignin varies according to the source (e.g., type of plant) and is so complex and varied that its molecular structure has never been completely described. As such, lignin monomers and polymers can have various molecular structures. Commonly known lignin monomers include p-coumaryl alcohol, coniferyl alcohol, and sinapyl alcohol.

The in situ catalytic reduction reaction can be considered a first hydrothermal reaction step, which produces a first reaction product that may also be referred to as a digestion product. At least a portion of the phenolics in the first reaction product can optionally be converted to hydrocarbon compounds by a second hydrothermal reaction step where the first reaction product is heated in the presence of molecular hydrogen and catalyst capable activating molecular hydrogen. The reaction product from the second hydrothermal reaction step can be referred to as a second reaction product. Unless otherwise specified, the term “reaction product” can refer to the first reaction product or the second reaction product. Optionally, at least some of the alcoholic component in the first reaction product, such as glycol or triol, including carbonyls, can also be converted to monohydric alcohol in the second hydrothermal reaction step.

Unconverted phenolics—phenolics that have not been converted to hydrocarbons (meaning compounds that still contain a hydroxyl group bonded to an aromatic hydrocarbon group)—may be recycled or returned to at least one of the first hydrothermal reaction step and the second hydrothermal reaction step. The phenols may serve as part of the digestion solvent in the first hydrothermal reaction step, and/or the phenols may be further converted to hydrocarbons in the second hydrothermal reaction step. It has been found that, if employed, a second hydrothermal reaction step having a low phenolics concentration provides better yields of hydrocarbons from phenols, than one with a high concentration of phenolics, which limits the amount of water and other mono-oxygenated compounds available in the reaction. That is, lignin reversion is better when the concentration of phenolics in the reaction content of the second hydrothermal reaction step is low versus when the phenolics concentration is high. For instance, low phenolics concentration in the second hydrothermal reaction step is a concentration of up to 50% by weight based on the total weight of the content of the second hydrothermal reaction step. Non-limiting illustrative phenolics concentrations of the reaction content in the second hydrothermal reaction step can be in a range of about 0.1% and up to 50% by weight, and any amount in between, including up to 45%, up to 40%, up to 35%, up to 30%, up to 25%, up to 20%, up to 15%, up to 10%, or up to 5% by weight, based on the total content weight of the second hydrothermal reaction step. It is understood that the referenced any phenolics concentration amount in between 0.1 wt % to 50 wt % includes at least 5 wt %, at least 10 wt %, at least 15 wt %, at least 20 wt %, at least 25 wt %, at least 30 wt %, at least 35 wt %, at least 40 wt %, at least 45 wt %, or at least 50 wt %.

Lignin reversion, including conversion of lignin to phenols and/or conversion of phenolics to hydrocarbon compounds, can be improved if the conversion is accomplished in the presence of water. That is the overall phenolics concentration in the second hydrothermal reaction step of 50% or less by weight of the content of the second hydrothermal reaction step can be achieved at least in part with water. For instance, the concentration of water in the second hydrothermal reaction step can be at least 10% by weight based on the total weight of the content of the second hydrothermal reaction step. Non-limiting illustrative water concentration of the reaction content in the second hydrothermal reaction step can be at least 10%, at least 15%, at least 20%, at least 25%, at least 30%, at least 35%, or at least 40% by weight, based on the total content weight of the second hydrothermal reaction step. It is realized that the concentrations mentioned for the first or second hydrothermal reaction content are not limited to single liquid phase. Instead, the concentrations provided correspond to the concentrations of water averaged over all phases present in the respective reactor, where two or sometimes three or more liquid phases may be observed. Additional details regarding systems and methods related to lignin reversion can be found in U.S. application Ser. No. 14/868,426, filed on Sep. 29, 2015, claiming priority to application No. 62/058,440, filed on Oct. 1, 2014, entitled METHODS AND SYSTEMS FOR PROCESSING CELLULOSIC BIOMASS, the disclosure of which is incorporated by reference in its entirety.

At least a portion of the first and/or second reaction product can be provided to a separation zone that provides for liquid-liquid extraction of the reaction product and product recovery of the phases independent of each other. Optionally, prior to being provided to the separation zone, excess gas in the reaction product can be removed. Liquid-liquid extraction employs an extraction solvent to extraction various compounds from the aqueous phase to provide an aqueous stream containing the aqueous phase and a non-aqueous stream containing the extracted compounds, solvent, and remaining portion of the reaction product not in the aqueous stream. At least a portion of the extraction solvent used in the liquid-liquid extraction comes from the reaction product itself, particularly from a higher hydrocarbon product or a distillate fraction thereof. Any suitable liquid-liquid extraction methods and systems capable of partitioning the reaction product into the aqueous stream and the non-aqueous stream can be used. These methods can be operated in batch mode, semi-batch mode, continuous mode, or any combination thereof.

Suitable types of apparatus that can be employed in the liquid-liquid extraction include stage-type extractors or differential extractors. An example of a stage-type extractor is a mixer-settler apparatus that typically includes a mixing tank coupled to a settling vessel to allow the phases to coalesce. In general, a mixer-settler apparatus can be used in batch mode, or a plurality of mixer-settler apparatus can be staged to operate in a continuous or semi-continuous mode. Another stage-type extractor is a centrifugal contactor. Various types of differential extractors (also known as “continuous contact extractors,”) that are also suitable include, but are not limited to, centrifugal contactors and contacting columns such as tray columns, spray columns, packed towers, rotating disc contactors and pulse columns.

The liquid-liquid extraction process can also involve apparatus that allows or facilitates coalescing of the phases, including a horizontal settling tank, one or more weirs, centrifuge- or hydroclone-type device, a fiber coalesce mat or a packed bed coalescer.

The content of the aqueous stream (which may also be referred to as the raffinate) exiting the liquid-liquid extraction depends on the level or degree of extraction, ranging from low to high. It is understood that the level of extraction can range anywhere between the low and high extremes. If the level of extraction is relatively low, the aqueous stream can still comprise compounds that have some miscibility with water, including light compounds, middle-boiling compounds, and heavy compounds, as well as water-soluble salts, ash, tar, and other waste. Light compounds include those with a normal boiling point of less than 150 degrees C., including monohydric alcohols, aldehydes, ketones, acids, and any combination thereof. Middle-boiling compounds, such as those with a normal boiling point of 150 to 350 degrees C., which can be present in the aqueous stream include glycol and phenols. Heavy compounds include those with a normal boiling point of greater than 350 degrees C. The light compounds and middle-boiling compounds can be recovered from the aqueous stream for further handling, including further processing of light compounds into a fuels product and recycling of middle-boiling compounds as a solvent.

Liquid-liquid extraction typically does not provide sharp cut-offs between the water-miscible compounds in the aqueous stream and non-water miscible compounds in the non-aqueous stream. The amount of compounds extracted from the aqueous phase depends on various factors as will be apparent to one of ordinary skill in the art, including the type of extraction and the type of extractor apparatus, the type of solvent and the solvent ratio.

If the level of extraction is relatively high, the aqueous stream may comprise only trace amounts of these compounds because the compounds have been extracted from the aqueous phase, and they exit the liquid-liquid extraction in the non-aqueous stream. As such, the extracted compounds may be recovered in subsequent handling of the non-aqueous stream, such as distillation. In such a circumstance, it may not be cost effective to subject the aqueous phase to a product recovery step, such as flashing. Consequently, the aqueous phase may be treated as waste in various manners known to one of ordinary skill, including those mentioned in the present disclosure.

The non-aqueous stream comprises a minor portion of water in the reaction product in the liquid-liquid extraction unit. While the non-aqueous stream can still contain some water, it is referred to as “non-aqueous” to distinguish it from the aqueous stream that contains substantially more water. In addition to the small amount of water, the non-aqueous stream also comprises a major portion of middle-boiling compounds and a smaller amount of light compounds due to the miscibility of certain light compounds with the non-aqueous phase(s). After liquid-liquid extraction, the non-aqueous stream can be distilled into various product fractions, including an overhead fraction comprising light compounds, a middle fraction comprising middle-boiling compounds, including cyclic alcohols, phenols, and lignin oligomers, and a bottom fraction comprising the heaviest of compounds in the reaction product, which are often regarded as waste or used as utility fuel. Additionally or alternatively, the non-aqueous stream can be distilled into more than one middle fraction.

The light compounds recovered from the distillation of the non-aqueous stream can be further processed into a higher hydrocarbon product or fuels product. A middle fraction can be recycled to at least one of the first hydrothermal reaction step and the second hydrothermal reaction step. For instance, various compounds in the middle fraction(s), including phenols, can serve as solvent in the first hydrothermal reaction step. Additionally or alternatively, various compounds in the middle fraction(s), including phenols and lignin oligomers, can be further hydrotreated in the second hydrothermal reaction step. For instance, phenols in the middle fraction(s) can get converted to hydrocarbons in the second hydrothermal reaction step, and lignin oligomers in the middle fraction(s) can be further reverted to phenols in the second hydrothermal reaction step, particularly in the presence of water. While lignin oligomers can be provided to the first hydrothermal reaction step, it is not preferred.

As described, at least a portion of the extraction solvent in the liquid-liquid extraction process can come from products recovered from the further processing reaction product or a distillation fraction thereof. Any suitable amount of recycled extraction solvent can be provided. In some circumstances, the amount of extraction solvent used in the liquid-liquid extraction process is a ratio of the reaction product in the liquid-liquid extraction process. For instance, the extraction solvent can be provided at a ratio in a range of 1 part solvent to 9 parts reaction product to 9 parts solvent to 1 part reaction product. Non-limiting illustrative ratios include 1 part solvent to 9 parts reaction product (1:9), 1 part solvent to 8 parts reaction product (1:8), 1 part solvent to 7 parts reaction product (1:7), 1 part solvent to 6 parts reaction product (1:6), 1 part solvent to 5 parts reaction product (1:5), 1 part solvent to 4 parts reaction product (1:4), 1 part solvent to 3 parts reaction product (1:3), 1 part solvent to 2 parts reaction product (1:2), 1 part solvent to 1 part reaction product (1:1), 2 parts solvent to 1 part reaction product (2:1), 3 parts solvent to 1 part reaction product (3:1), 4 parts solvent to 1 part reaction product (4:1), 5 parts solvent to 1 part reaction product (5:1), 6 parts solvent to 1 part reaction product (6:1), 7 parts solvent to 1 part reaction product (7:1), 8 parts solvent to 1 part reaction product (8:1), 9 parts solvent to 1 part reaction product (9:1), 10 parts solvent to 1 part reaction product (10:1), and any combination thereof.

As described, phenols serving as solvent in the first hydrothermal reaction step can come from products recovered from the reaction product. Any suitable amount of recycled phenols—phenols coming from the reaction product—can be provided. In some circumstances, the amount of phenols provided to the first hydrothermal reaction step is a ratio of the amount of lignin provided thereto. For instance, phenols can be provided at a ratio of at least 1 part phenols to 2 parts lignin (1:2), including a ratio in a range of 1 part phenols to 2 parts lignin (1:2) to 10 parts phenols to 1 part lignin (10:1), or any ratio in between. Non-limiting illustrative ratios include at least 1 part phenols to 1 part lignin (1:1), 2 parts phenols to 1 part lignin (2:1), 3 parts phenols to 1 part lignin, 4 parts phenols to 1 part lignin (4:1), 5 parts phenols to 1 part lignin (5:1), 6 parts phenols to 1 part lignin (6:1), 7 parts phenols to 1 part lignin (7:1), 8 parts phenols to 1 part lignin (8:1), 9 parts phenols to 1 part lignin (9:1), 10 parts phenols to 1 part lignin (10:1), and any combination thereof. It is understood that lignin is provided as a component of the biomass feed, or via recycle of a process stream containing lignin.

If a second hydrothermal reaction step is present, glycols, phenols, and/or lignin oligomers recovered from the reaction product can be provided to it. Any suitable amount of recycled phenols and/or lignin oligomers can be provided consistent with the low phenolics concentration of up to 50% by weight based on the total weight of the content of the second hydrothermal reaction step discussed above. The term “recycled” refers to compounds recovered from the reaction product.

Optionally, the non-aqueous stream can be subject to one or more lignin depolymerization reactions prior to distillation to reduce the viscosity of the non-aqueous stream, thereby improving the separation of the product fractions during distillation. Any suitable lignin depolymerization reaction can be used. In general, lignin depolymerization reactions revert high molecular weight lignin oligomers to lower molecular weight compounds, such as lighter lignin oligomers and/or lignin monomers, which results in viscosity reduction. Depolymerization of lignin in the non-aqueous stream provides lignin monomers that facilitate phenolics separation from the lignin oligomers and even heavier compounds. The lignin monomers can become a cutting agent to reduce the viscosity of the high molecular weight lignin oligomers. The middle fraction comprising phenols and/or lignin oligomers distilled from the non-aqueous stream can also be provided to the lignin depolymerization reactions to further reduce the viscosity of the feed into the distillation unit, thereby enhancing product separation. It is recognized that this step may necessarily involve use of less water and optionally can be conducted in the absence of catalyst, such that higher temperatures (in excess of 300° C.) may be required for effective conversion of lignin to smaller fragments of lower viscosity via this step.

The following paragraphs provide additional details of the provided methods and systems for processing cellulosic biomass into a fuel product, particularly processing of a reaction product obtained from hydrothermal reaction of cellulosic biomass solids. As mentioned above, hydrothermal reaction of a cellulosic biomass can include in situ catalytic reduction, which involves heating of the cellulosic biomass and a digestion solvent in the presence of molecular hydrogen and a catalyst capable of activating the molecular hydrogen. Any type of suitable catalyst capable of activating hydrogen can be used in any reactor suitable for use with the selected catalyst(s) for the first and/or second hydrothermal reaction step. For example, at least one of the first and second hydrothermal reaction steps can be carried out using fluidly mobile catalyst particles that can be at least partially suspended in a fluid phase via gas flow, liquid flow, mechanical agitation, or any combination thereof in a reactor. Various conditions can be implemented so that the fluidly mobile catalyst particles do not get carried out of the reactor by the fluid flowing through the reactor. A reactor operating under these circumstances can be called an ebullating bed reactor in part because the catalyst particles remain in the reactor to form a catalytic bed. It is understood that one of ordinary skill in the art can select the various conditions to achieve an ebullating bed reactor. For instance, a suitable concentration of catalyst and/or catalyst size can be selected to obtain the desired reactor conditions.

On the other hand, the conditions can be modified so that the fluidly mobile catalyst particles flow with the biomass solids through the reactor. A reactor operating under these circumstances can be called a slurry reactor. Adequate catalyst distribution is desirable in a slurry reactor so that soluble carbohydrates formed during hydrothermal digestion may be intercepted and converted into more stable compounds before they have had an opportunity to significantly degrade, even under thermal conditions that otherwise promote their degradation.

If present, the second hydrothermal reaction step can also be carried out using a catalyst that does not comprise fluidly mobile catalyst particles. For example, the second hydrothermal reaction step can be carried out in a fixed bed reactor or a trickle bed reactor, which are known by one of ordinary skill in the art. For instance, during operation of a fixed bed or trickle bed reactor and fluid is flowing through the reactor, the height of the bed does not increase to greater than 10% as compared to when fluid is not flowing through the reactor.

If a reactor with a catalyst that does not comprise fluidly mobile catalyst particles is used in the second hydrothermal reaction step, a possible issue may be clogging of the bed by cellulosic particulates in the first reaction product from the digestion. As cellulosic biomass breaks apart during digestion, smaller and smaller particulates may be produced until only insoluble materials remain. Cellulosic particulates may also be present in native cellulosic biomass before digestion takes place. One way of handling cellulosic particulates can be use of a screen at a fluid outlet of the hydrothermal digestion unit to assist in maintaining the cellulosic fines therein. Periodic backflow or “backflush” of liquid or gas may be necessary to flush solids from this screen, to maintain fluid outflow. At a certain size, the cellulosic particulates may pass through the screen of the hydrothermal digestion unit and enter at least the reactor in the second reaction zone.

Another way to address cellulosic particulates, particularly ones that are sufficiently small to pass through screens, is the methods and systems disclosed in commonly owned U.S. Application Publication No. 2013/0152456 (“the '456 publication”), the disclosure of which is incorporated herein in its entirety. In general, the '456 publication discloses a solids separation unit to which the first reaction product can be routed to have at least a portion of the cellulosic particulates removed before it enters a reactor in the second reaction zone. The solids separation unit can comprise one or more filters, where at least one of the filters can be backflushed to remove cellulosic fines therefrom, while one or more of the other filters remain in fluid communication with an inlet of the reactor in the second reaction zone.

In some embodiments, catalysts capable of activating molecular hydrogen may comprise a metal such as, for example, Cr, Mo, W, Re, Mn, Cu, Cd, Fe, Co, Ni, Pt, Pd, Rh, Ru, Ir, Os, and alloys or any combination thereof, either alone or with promoters such as Au, Ag, Cr, Zn, Mn, Sn, Bi, B, O, and alloys or any combination thereof. In some embodiments, the catalysts and promoters may allow for various hydrothermal reactions, such as hydrogenation and hydrogenolysis reactions, to occur at the same time or in succession of one another. In some embodiments, such catalysts may also comprise a carbonaceous pyropolymer catalyst containing transition metals (e.g., Cr, Mo, W, Re, Mn, Cu, and Cd) or Group VIII metals (e.g., Fe, Co, Ni, Pt, Pd, Rh, Ru, Ir, and Os). In some embodiments, the foregoing catalysts may be combined with an alkaline earth metal oxide or adhered to a catalytically active support. In some or other embodiments, the catalyst capable of activating molecular hydrogen may be deposited on a catalyst support that is not itself catalytically active.

In some embodiments, the catalyst used in the first and/or second hydrothermal reaction step may comprise a poison-tolerant catalyst. As used herein the term “poison-tolerant catalyst” refers to a catalyst that is capable of activating molecular hydrogen without needing to be regenerated or replaced due to low catalytic activity for at least about 12 hours of continuous operation. Use of a poison-tolerant catalyst may be particularly desirable when reacting soluble carbohydrates derived from cellulosic biomass solids that have not had catalyst poisons removed therefrom. Catalysts that are not poison tolerant may also be used to achieve a similar result, but they may need to be regenerated or replaced more frequently than does a poison-tolerant catalyst.

Suitable poison-tolerant catalysts may include, for example, sulfided catalysts. In some or other embodiments, nitrided catalysts may be used as poison-tolerant catalysts. Sulfided catalysts suitable for activating molecular hydrogen are described in commonly owned Patent Application Publication US2012/0317872 and U.S. Pat. No. 8,921,629, each of which is incorporated herein by reference in its entirety.

The catalyst particles may have a particulate size of about 250 microns or less, about 100 microns or less, or about 10 microns or less. The minimum particulate size of the catalyst particles may be about 1 micron. The catalyst particles may comprise catalyst fines. As used herein, the term “catalyst fines” refers to solid catalysts having a nominal particulate size of about 100 microns or less. Catalyst fines may be generated from catalyst production processes, for example, during extrusion of solid catalysts. Catalyst fines may also be produced by grinding larger catalyst solids or during regeneration of catalyst solids. Suitable methods for producing catalyst fines are described in U.S. Pat. Nos. 6,030,915 and 6,127,229, each of which is incorporated herein by reference in its entirety. In some instances, catalyst fines may be intentionally removed from a solid catalyst production run, since they may be difficult to sequester in some catalytic processes. Techniques for removing catalyst fines from larger catalyst solids may include, for example, sieving or like size separation processes. When conducting in situ catalytic reduction reaction processes, such as those described herein, catalyst fines may be particularly well suited, since they can be easily fluidized and distributed in the interstitial pore space of the digesting cellulosic biomass solids.

Catalysts that are not particularly poison-tolerant may also be used in conjunction with the techniques described herein. Such catalysts may include, for example, Ru, Pt, Pd, or compounds thereof disposed on a solid support such as, for example, Ru on titanium dioxide or Ru on carbon. Although such catalysts may not have particular poison tolerance, they may be regenerable, such as through exposure of the catalyst to water at elevated temperatures, which may be in either a subcritical state or a supercritical state.

The catalysts used in conjunction with the processes described herein may be operable to generate molecular hydrogen. For example, in some embodiments, catalysts suitable for aqueous phase reforming (i.e., APR catalysts) may be used. Suitable APR catalysts may include, for example, catalysts comprising Pt, Pd, Ru, Ni, Co, or other Group VIII metals alloyed or modified with Re, Mo, Sn, or other metals. Thus, in some embodiments described herein, an external hydrogen feed may not be needed in order to effectively carry out the stabilization of soluble carbohydrates by a catalytic reduction reaction. However, in other embodiments, an external hydrogen feed may be used, optionally in combination with internally generated hydrogen. In yet other embodiments, the molecular hydrogen needed may be externally supplied to the cellulosic biomass solids. If external hydrogen is provided, the molecular hydrogen may be supplied as an upwardly directed fluid stream. Benefits of supplying an upwardly directed fluid stream are described herein.

The digestion solvent provided to the first reaction zone may comprise an organic solvent. In various embodiments, the digestion solvent may comprise an organic solvent and water. Although any organic solvent that is at least partially miscible with water may be used in the digestion solvent, particularly advantageous organic solvents are those that can be directly converted into fuel blends and other materials without being separated from the alcoholic component. That is, particularly advantageous organic solvents are those that may be co-processed during downstream further processing reactions with the alcoholic component being produced. Suitable organic solvents in this regard may include, for example, ethanol, ethylene glycol, propylene glycol, glycerol, phenols, and any combination thereof. Other suitable organic solvents may include sugar alcohols, for example.

In some embodiments, the digestion solvent may further comprise a small amount of a monohydric alcohol. The presence of at least some monohydric alcohols in the digestion solvent may desirably enhance the digestion and/or the catalytic reduction reactions being conducted therein. For example, inclusion of about 1% to about 5% by weight monohydric alcohols in the digestion solvent may desirably maintain catalyst activity due to a surface cleaning effect. At higher concentrations of monohydric alcohols, bulk solvent effects may begin to predominate. In some embodiments, the digestion solvent may comprise about 10 wt. % or less monohydric alcohols, with the balance of the digestion solvent comprising water and another organic solvent. In some embodiments, the digestion solvent may comprise about 5 wt. % or less monohydric alcohols, or about 4% or less monohydric alcohols, or about 3% or less monohydric alcohols, or about 2% of less monohydric alcohols, or about 1% or less monohydric alcohols. Monohydric alcohols present in the digestion solvent may arise from any source. In some embodiments, the monohydric alcohols may be formed as a co-product with the alcoholic component being formed by the catalytic reduction reaction. In some or other embodiments, the monohydric alcohols may be formed by a subsequent catalytic reduction of the initially produced alcoholic component and thereafter returned to the cellulosic biomass solids. In still other embodiments, the monohydric alcohols may be sourced from an external feed that is in flow communication with the cellulosic biomass solids.

In some embodiments, the digestion solvent may comprise between about 1% water and about 99% water, with the organic solvent comprising the balance of the digestion solvent composition. Although higher percentages of water may be more favorable from an environmental standpoint, higher quantities of organic solvent may more effectively promote hydrothermal digestion due to the organic solvent's greater propensity to solubilize carbohydrates and promote catalytic reduction of the soluble carbohydrates. In some embodiments, the digestion solvent may comprise about 90% or less water by weight. In other embodiments, the digestion solvent may comprise about 80% or less water by weight, or about 70% or less water by weight, or about 60% or less water by weight, or about 50% or less water by weight, or about 40% or less water by weight, or about 30% or less water by weight, or about 20% or less water by weight, or about 10% or less water by weight, or about 5% or less water by weight.

The further processing zone may generally comprise a condensation reaction, often conducted in the presence of a condensation catalyst, in which the alcoholic component or a product formed therefrom is condensed with another molecule to form a higher molecular weight compound, i.e., higher hydrocarbon. As used herein, the term “condensation reaction” will refer to a chemical transformation in which two or more molecules are coupled with one another to form a carbon-carbon bond in a higher molecular weight compound, usually accompanied by the loss of a small molecule such as water or an alcohol. An illustrative condensation reaction is the Aldol condensation reaction, which will be familiar to one having ordinary skill in the art.

A portion of the higher hydrocarbon products from the condensation reaction may be provided to full distillation or multi-distillation to obtain various product fractions. Optionally, water may be removed from the higher hydrocarbon products prior to distillation. In an embodiment, the product fractions from the high hydrocarbon products stream can include at least the lighter fractions ranging from those containing 4 carbon atoms to those containing 11 or 12 carbon atoms. It has an initial boiling point at atmospheric pressure of about 35° C. (95° F.) and a final boiling point of about 200° C. (395° F.), primarily may be separated for gasoline use. The lighter fractions tend to be rich in aromatics content (“aromatics-rich stream” or “aromatics-rich hydrocarbon”) The aromatics-rich hydrocarbon fraction may contain aromatics content of greater than 10 wt %, preferably at least 25 wt %, more preferably at least 40 wt % aromatics such as, for example, benzenes, toluenes, dimethyl benzenes, trimethylbenzenes, and the like. In an embodiment, the product fractions from the high hydrocarbon products stream can include at least the moderate fractions ranging between 200° C. (392° F.) and 350° C. (662° F.) at atmospheric pressure, resulting in a mixture of carbon chains that typically contain between 8 and 21 carbon atoms per molecule, primarily may be separated for diesel use. The moderate fractions tend to be lower in aromatics content (“low aromatics hydrocarbon”). In some embodiment, the low aromatics content hydrocarbon fraction may contain aromatics content of at most 10 wt %. Anther moderate fractions, e.g. C7-C14, may be separated for use as kerosene, e.g. for jet fuel use. The heaviest fractions may be used as lubricants or may be cracked to produce additional fractions for use in gasoline, kerosene and/or diesel fractions. Such lighter fraction or any of the medium fractions can be used as the extractive solvent in the liquid-liquid extraction in the separation zone as at least a portion of the higher hydrocarbon product. The aromatics-rich hydrocarbon may be a particular efficient liquid-liquid extraction solvent.

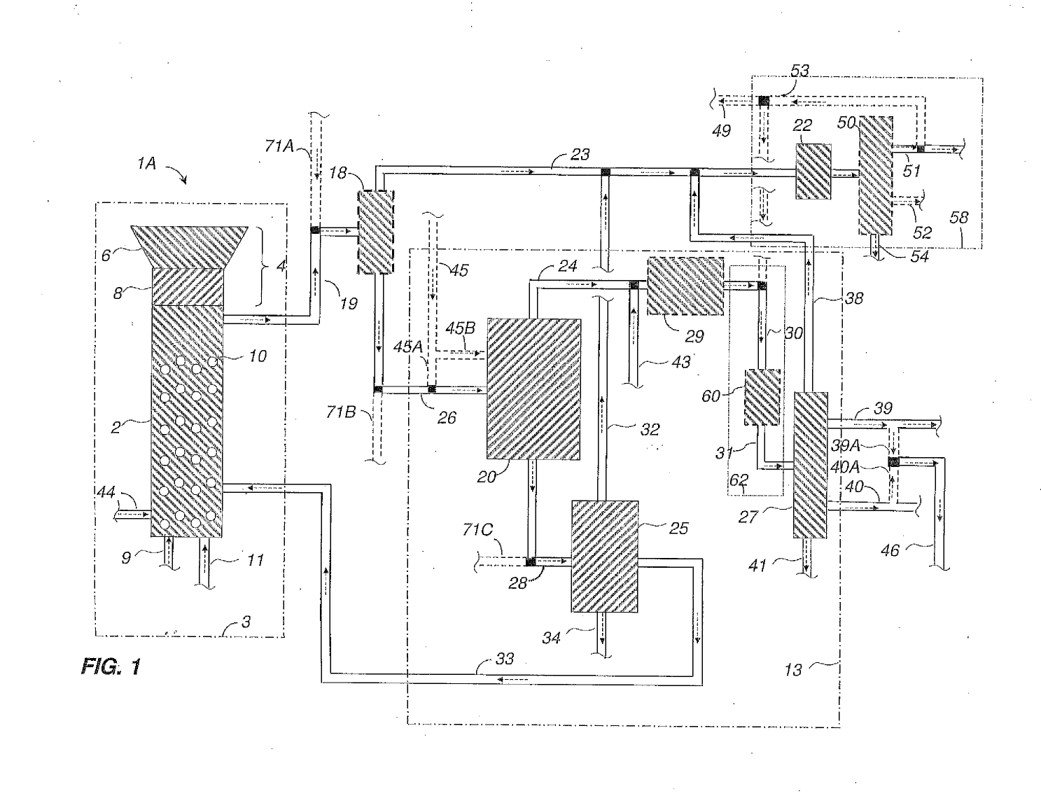

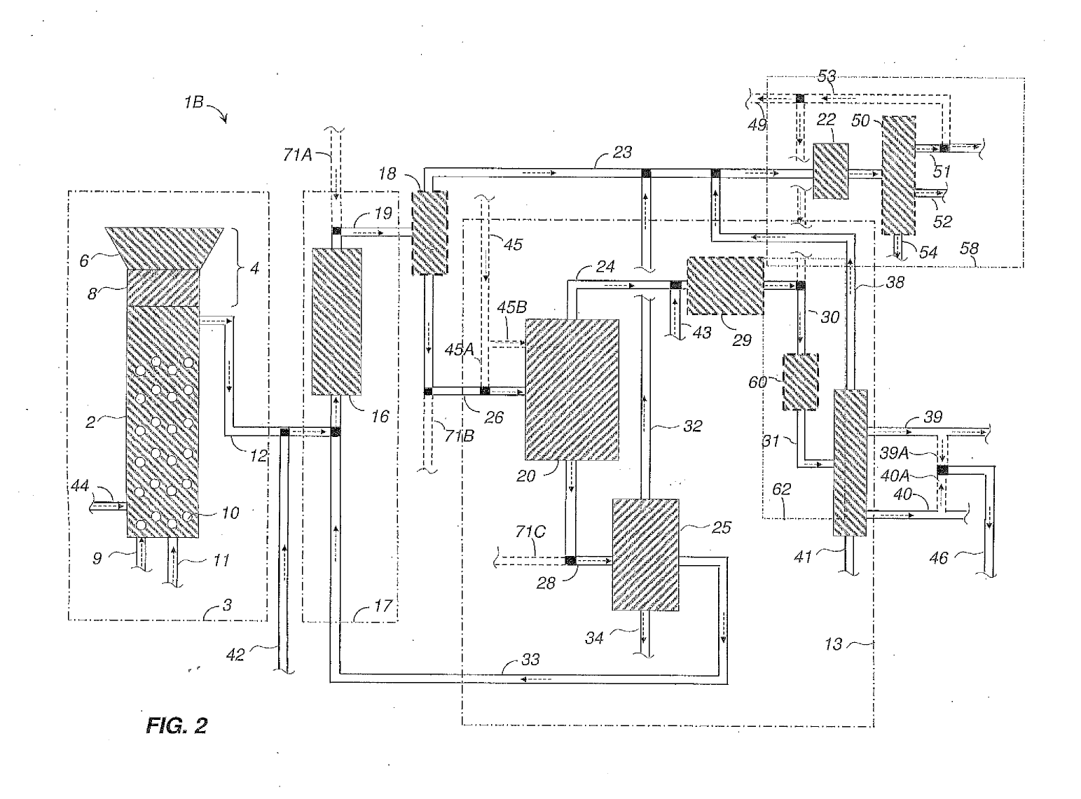

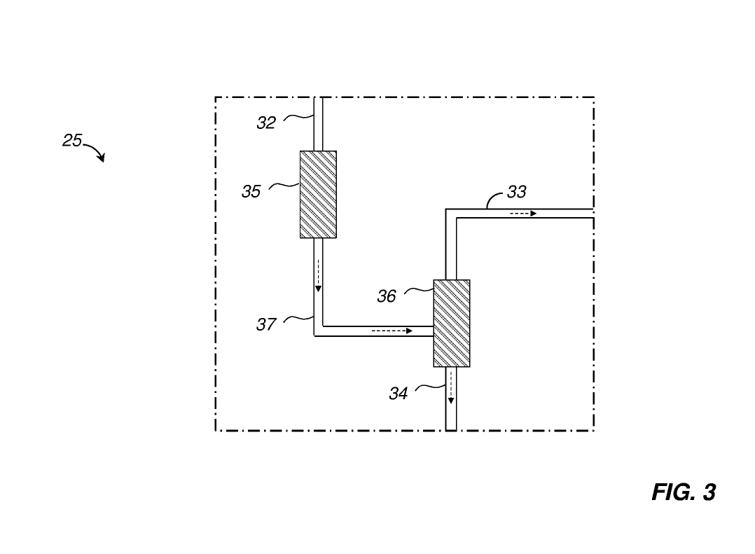

Various illustrative embodiments will be further described with reference to FIGS. 1-3. In particular, FIGS. 1-2 show illustrative embodiments of biomass processing system 1, specifically system 1A in FIG. 1 and system 1B in FIG. 2. FIG. 3 shows an embodiment of aqueous separation zone 25 that can be employed in a separation zone in a system or method to process cellulosic biomass as provided by the present disclosure, such as in separation zone 13 of system 1 depicted in FIGS. 1-2.

Referring to FIGS. 1-2, hydrothermal digestion unit 2 in first reaction zone 3, which can also be referred to as digestion zone 3, contains a first reaction content comprising cellulosic biomass, a catalyst capable of activating molecular hydrogen, a digestion solvent, and molecular hydrogen. As further discussed below, the first reaction content subsequent to the initial materials provided to hydrothermal digestion unit 2 can further comprise a recycled phenolic portion that may be part of the digestion solvent. While FIGS. 1-2 show one hydrothermal digestion unit 2 in digestion zone 3, it is understood that digestion zone 3 can comprise any suitable number of hydrothermal digestion unit coupled to one another (e.g., in fluid communication with one another), such as at least two, three, four, five, six, or more hydrothermal digestion units. The catalyst capable of activating molecular hydrogen provided to hydrothermal digestion unit 2 preferably comprises fluidly mobile catalyst particles 10. For instance, hydrothermal digestion unit 2 can be a slurry reactor or an ebullating bed reactor. In the interest of clarity, the cellulosic biomass, digestion solvent, and molecular hydrogen in hydrothermal digestion unit 2 have not been depicted. If reaction zone 3 has more than one hydrothermal digestion units, these units can be any combination of a slurry reactor and/or an ebullating bed reactor.

The reaction content in hydrothermal digestion unit 2 is heated to form a first reaction product comprising phenols and an alcoholic component. The phenols are derived from lignin in the cellulosic biomass, and the alcoholic component is formed from soluble carbohydrates derived from cellulosic biomass. Heating of the first reaction content provides for in situ catalytic reduction where digestion of the cellulosic biomass and catalytic reduction of soluble carbohydrates takes place in the same reactor. The reaction content in hydrothermal digestion unit 2 is heated to a temperature that may be in a range of about 190 to 290 degrees C., such as in a range of about 200 to 275 degrees C., or such as in a range of about 190 to about 260 degrees C. For instance, the reaction content in hydrothermal digestion unit 2 can be heated to about 190, 195, 200, 205, 210, 215, 220, 225, 230, 235, 240, 245, 250, 255, or 260 degrees C.

The heating of the first reaction content in hydrothermal digestion unit 2 is preferably performed under a pressurized state. As used herein, the term “pressurized state” refers to a pressure that is greater than atmospheric pressure (1 bar). For example, hydrothermal digestion unit 2 may have a pressure of at least about 30 bar, such as at least about 45 bar, at least about 60 bar, at least about 75 bar, at least about 90 bar, at least about 100 bar, at least about 110 bar, at least about 120 bar, or at least about 130. Hydrothermal digestion unit 2 may have a pressure of at most about 450 bar, such as at most about 330 bar, at most about 200 bar, at most about 175 bar, at most about 150 bar, or at most about 130 bar. As such, hydrothermal digestion unit 2 may have a pressure in a range of about 30 to 450 bar, such as a range of about 45 and 330 bar or in a range of about 75 to 130 bar. Hydrogen is preferably added to achieve the desired total pressure of hydrothermal digestion unit 2. For instance, hydrogen partial pressure of greater than 5 bar, greater than 10, or greater than 25 bar can be provided to hydrothermal digestion unit 2 to achieve the desired total pressure. Additional pressure may occur from vaporization of a portion of the water and volatile compounds of the reaction content. Heating of the digestion solvent in hydrothermal digestion unit 2 in a pressurized state may allow the normal boiling point of various compounds of the digestion solvent to be exceeded, thus allowing a predominately liquid state to be maintained, thereby allowing the rate of hydrothermal digestion to be increased relative to lower temperature digestion processes.

The reaction content in hydrothermal digestion unit 2 may be heated for at least 30 minutes and up to 10 hours, most typically between 120 minutes to 300 minutes. For example, digestion may be carried out for at least 30 minutes, at least 60 minutes, at least 120 minutes, at least 180 minutes, at least 240 minutes, at least 300 minutes, at least 360 minutes, at least 420 minutes, at least 480 minutes, at least 540 minutes, or at least 600 minutes. Digestion may be carried out at most 600 minutes, at most 540 minutes, at most 480 minutes, at most 420 minutes, at most 360 minutes, at most 300 minutes, at most 240 minutes, at most 180 minutes, at most 120 minutes, at most 60 minutes, or at most 30 minutes.

Referring to FIGS. 1-2, hydrothermal digestion unit 2 may be charged with a desired amount of catalyst particulates 10, while cellulosic biomass solids are continuously or semi-continuously added thereto at a more frequent rate, thereby allowing hydrothermal digestion to take place in a continual manner. Catalyst solids may be periodically withdrawn and replaced with fresh catalyst, to compensate for degradation of catalyst during operation, or poisoning via components in the biomass feed. Cellulosic biomass may be introduced to hydrothermal digestion unit 2 in the first reaction zone via solids introduction mechanism 4. As used herein, the term “continuous addition” and grammatical equivalents thereof will refer to a process in which cellulosic biomass solids are added to a hydrothermal digestion unit in an uninterrupted manner without fully depressurizing the hydrothermal digestion unit. As used herein, the term “semi-continuous addition” and grammatical equivalents thereof will refer to a discontinuous, but as-needed, addition of cellulosic biomass solids to a hydrothermal digestion unit without fully depressurizing the hydrothermal digestion unit. That is, fresh cellulosic biomass solids may be added to hydrothermal digestion unit 2 on a continual or an as-needed basis in order to replenish cellulosic biomass solids that have been digested to form soluble carbohydrates.

Solids introduction mechanism 4 may comprise loading mechanism 6 and pressure transition zone 8, which may continuously or periodically elevate the cellulosic biomass from atmospheric pressure to a pressure near that of the operating pressure of hydrothermal digestion unit 2, particularly when hydrothermal digestion unit 2 is in a pressurized state. This allows for continuous or semi-continuous introduction of cellulosic biomass to take place without fully depressurizing hydrothermal digestion unit 2. That is, the cellulosic biomass solids may be continuously or semi-continuously added to the hydrothermal digestion unit while the hydrothermal digestion unit is in a pressurized state. Without the ability to introduce fresh cellulosic biomass to a pressurized hydrothermal digestion unit, depressurization and cooling of the hydrothermal digestion unit may take place during biomass addition, significantly reducing the energy- and cost-efficiency of the biomass conversion process.

Pressure transition zone 8 may comprise one or more suitable pressurization zones for pressurizing and introducing cellulosic biomass solids to hydrothermal digestion unit 2. Such suitable pressurization zones are described in more detail in commonly owned United States Patent Application Publications 2013/0152457 and 2013/0152458, and incorporated herein by reference in their entirety. Another suitable example of an illustrative pressure transition zone is described in more detail in commonly owned U.S. Patent Application No. 62/058,177, filed on Oct. 1, 2014, entitled Systems and Methods for Providing Feed Material to a Pressurized System. Suitable pressurization zones described therein may include, for example, pressure vessels, pressurized screw feeders, one or more pumps capable of pumping solids and solids slurries, and the like. Multiple pressurization zones may be connected in series to increase the pressure of the cellulosic biomass solids in a stepwise manner.

In various embodiments, soluble carbohydrates produced from cellulosic biomass solids may be converted into a reaction product comprising a glycol via a catalytic reduction reaction mediated by a catalyst that is capable of activating molecular hydrogen. As described in commonly owned United States Patent Application Publications 20140121420 and 20140128639, entitled “Methods for Production and Processing of a Glycol Reaction Product Obtained from Hydrothermal Digestion of Cellulosic Biomass Solids” and “Methods for Conversion of a Glycol Reaction Product Obtained from Hydrothermal Digestion of Cellulosic Biomass Solids Into a Dried Monohydric Alcohol Feed,” and incorporated herein by reference in its entirety, production of glycols may present several process advantages, particularly with regard to downstream further processing reactions. In other aspects, formation of monohydric alcohols may be more desirable.

Referring to FIGS. 1-2, catalyst particulates 10 are capable of activating molecular hydrogen. At least a portion of catalyst particulates 10 may be distributed in the cellulosic biomass, particularly in hydrothermal digestion unit 2. If in situ catalytic reduction is carried out, effective distribution of catalyst particulates 10 throughout cellulosic biomass solids for in situ catalytic reduction reaction is desired. This may be achieved by using fluid flow to convey catalyst particulates 10 into the interstitial spaces within a charge of cellulosic biomass solids. As used herein, the terms “distribute,” “distribution,” and variants thereof refer to a condition in which catalyst particulates are present at all heights of a charge of cellulosic biomass. No particular degree of distribution is implied by use of the term “distribute” or its variants. Catalyst distribution may comprise a substantially homogeneous distribution, such that a concentration of catalyst particulates is substantially the same at all heights of a cellulosic biomass charge. Catalyst distribution may comprise a heterogeneous distribution, such that different concentrations of catalyst particulates are present at various heights of the cellulosic biomass charge. When a heterogeneous distribution of catalyst particulates is present, a concentration of catalyst particulates in the cellulosic biomass solids in hydrothermal digestion unit 2 may increase from top to bottom or decrease from top to bottom. In some embodiments, a heterogeneous distribution may comprise an irregular concentration gradient.

Catalyst particulates 10 may be conveyed into the cellulosic biomass solids in hydrothermal digestion unit 2 for distribution using fluid flow from any direction. In particular, at least a portion of catalyst particulates 10 may be conveyed by upwardly directed fluid flow, or at least that upwardly directed fluid flow is present. For instance, catalyst particulates 10 may be supplied through fluid inlet line 9 as shown in FIGS. 1-2. Such upwardly directed fluid flow may promote expansion of the cellulosic biomass solids and disfavor gravity-induced compaction that occurs during their addition and digestion. In addition, when upwardly directed fluid flow is present, there may be a reduced need to utilize mechanical stirring or like mechanical agitation techniques that might otherwise be needed to obtain an adequate catalyst distribution.

Suitable techniques for using fluid flow to distribute catalyst particulates 10 within cellulosic biomass solids are described in commonly owned United States Patent Application Publications 2014/0005445 and 2014/0005444, and incorporated herein by reference in its entirety. As described therein, cellulosic biomass solids may have at least some innate propensity for retaining catalyst particulates 10 being conveyed by fluid flow, and at least a portion of the cellulosic biomass solids may be sized to better promote such retention. In addition, using fluid flow, particularly upwardly directed fluid flow, to force catalyst particulates 10 to actively circulate through a charge of digesting cellulosic biomass solids may ensure adequate catalyst distribution as well as advantageously reduce thermal gradients that may occur during hydrothermal digestion. As a further advantage, active circulation of catalyst particulates 10 may address the problem created by the production of cellulosic biomass fines, since they may be co-circulated with catalyst particulates for continued digestion to take place in hydrothermal digestion unit 2.

The upwardly directed fluid flow may comprise a gas stream, a liquid stream, or any combination thereof. Also, the upwardly directed fluid flow may comprise one upwardly directed fluid stream, or two upwardly directed fluid streams, or three upwardly directed fluid streams, or four upwardly directed fluid streams, or five upwardly directed fluid streams.

At least some of the one or more upwardly directed fluid streams may contain catalyst particulates at its source. That is, the fluid stream(s) may comprise a stream of catalyst particulates. The one or more upwardly directed fluid streams may convey catalyst particulates therein. In other circumstances, the one or more upwardly directed fluid streams may not contain catalyst particulates at its source, but they may still fluidize catalyst particulates located in or near the cellulosic biomass solids.

The one or more upwardly directed fluid streams may comprise a gas stream. For example, a gas stream being used for upwardly directed fluid flow may comprise a stream of molecular hydrogen. Steam, or an inert gas such as nitrogen, for example, may be used in place of or in addition to a stream of molecular hydrogen. Up to about 40% steam may be present in the fluid stream.

The one or more upwardly directed fluid streams may comprise a liquid stream, particularly when it is not necessarily desired to maintain catalyst particulates in the cellulosic biomass solids and/or a gas stream alone is insufficient to distribute catalyst particulates, for example. Unlike a gas stream, a liquid stream may convey catalyst particulates through the cellulosic biomass solids, add to the liquid head surrounding the cellulosic biomass solids, and eventually spill over. In other circumstances, catalyst fluidization may be incomplete, and a liquid stream may still not convey catalyst particulates completely through the cellulosic biomass solids before the liquid head spills over.

As such, in certain instances, at least a portion of the liquid head may be circulated through the cellulosic biomass solids. Suitable hydrothermal digestion units configured for circulating a liquid phase therethrough, such as hydrothermal digestion unit 2 depicted in FIGS. 1-2, are described in commonly owned United States Patent Application Publication 2014/0004015 and incorporated herein by reference in its entirety. Specifically, hydrothermal digestion unit 2 may comprise a fluid circulation loop through which the liquid phase and optionally catalyst particulates 10 are circulated for distribution in the cellulosic biomass solids.

Another way to distribute catalyst particulates 10 is to convey at least a portion comprising phenols in hydrothermal digestion unit 2 to a point above at least a portion of the cellulosic biomass solids and release that portion. Catalyst particulates 10 can have the tendency to accumulate and agglomerate in larger particles in presence of phenols, particularly if the phenols aggregate to form a phenols-rich liquid phase, which contains a major portion of the phenols in unit 2. The formation of the phenols-rich liquid phase provides a catalyst-rich fraction, which contains a major portion of catalyst in unit 2, above cellulosic biomass solids in hydrothermal digestion unit 2, which can act to release catalyst particulates for downward percolation through the cellulosic biomass solids. Techniques for downward percolation of catalyst particulates and phenols are described in commonly owned U.S. Patent Application Publication No. 2014/0117276, entitled “Methods and Systems for Distributing a Slurry Catalyst in Cellulosic Biomass Solids” and U.S. Patent Publication No. 2014/0174432, the disclosures of which are incorporated herein by reference in their entirety.

In the embodiment of system 1 shown in FIG. 1, the first reaction product from hydrothermal digestion unit 2 may be provided to separation zone 13. FIG. 2 shows another embodiment of system 1—system 1B—where the first reaction product is provided via line 12 to phenolics conversion unit 16 in a second reaction zone, zone 17. While FIG. 2 shows one phenolics conversion unit 16 in reaction zone 17, it is understood that reaction zone 17 can comprise any suitable number of reactors coupled to one another (e.g., in fluid communication with one another), such as at least two, three, four, five, six, or more phenolics conversion units. Phenolics conversion unit 16 produces a second reaction product that is provided to separation zone 13. A portion of the diols and glycols present in digestion product 12 can also be converted to desired mono-oxygenated intermediates via conversion unit 16.

As shown in FIG. 2, the first reaction product is optionally part of a second reaction content that is provided to phenolics conversion unit 16. The second reaction content further comprises catalyst capable of activating molecular hydrogen, and molecular hydrogen. As further discussed below, the second reaction content subsequent to the initial materials provided to phenolics conversion unit 16 can also further comprise a phenolics portion. The second reaction content is heated in phenolics conversion unit 16 to form a second reaction product comprising unconverted phenolics, hydrocarbons converted from phenolics. Optionally, the second reaction product can also comprise monohydric alcohols converted from triols and glycol in the alcoholic component in the first reaction product.

Referring to FIG. 2, heating of the second reaction content in phenolics conversion unit 16 hydrotreat compounds in the first reaction product that are hydrotreatable but had not been hydrotreated in hydrothermal digestion unit 2. In particular, heating of the second reaction content provides for conversion of at least a portion of the phenolics in phenolics conversion unit 16 to hydrocarbons, and optionally, conversion of at least a portion of triol and glycol to monohydric alcohol. The reaction content in phenolics conversion unit 16 is heated to a temperature that may be in a range of about 210 to 300 degrees C., such as in a range of 270 to 290 degrees C. or at least 270 degrees C. For instance, the reaction content in phenolics conversion unit can be heated to about 210, 215, 220, 225, 230, 235, 240, 245, 250, 255, 260, 265, 270, 275, 280, 285, 290, 295, or 300 degrees C. In a particular embodiment, a temperature of the second reaction product at an outlet of phenolics conversion unit 16 is greater than a temperature of the first reaction product at an outlet of hydrothermal digestion unit 2. The hydrotreating of the second reaction content in phenolics conversion unit 16 can also provide for regeneration of catalyst particulates accumulated in the first reaction product, if they are present.

In FIG. 2, phenolics conversion unit 16 is preferably in a pressurized state. For example, phenolics conversion unit 16 may have a pressure of at least about 30 bar, such as at least about 45 bar, at least about 60 bar, at least about 75 bar, at least about 90 bar, at least about 100 bar, at least about 110 bar, at least about 120 bar, or at least about 130. Phenolics conversion unit 16 may have a pressure of at most about 450 bar, such as at most about 330 bar, at most about 200 bar, at most about 175 bar, at most about 150 bar, or at most about 130 bar. As such, phenolics conversion unit 16 may have a pressure in a range of about 30 to 450 bar, such as a range of about 45 and 330 bar or in a range of about 75 to 130 bar. Hydrogen is preferably used to achieve the desired total pressure of phenolics conversion unit 16. For instance, hydrogen partial pressure of greater than 5 bar, greater than 10, or greater than 25 bar can be provided to phenolics conversion unit 16 to achieve the desired total pressure.

Referring to FIG. 2, the reaction content in phenolics conversion unit 16 may be heated for at least 30 minutes and up to 10 hours, such as 120 minutes to 300 minutes. For example, digestion may be carried out for at least 30 minutes, at least 60 minutes, at least 120 minutes, at least 180 minutes, at least 240 minutes, at least 300 minutes, at least 360 minutes, at least 420 minutes, at least 480 minutes, at least 540 minutes, or at least 600 minutes. Digestion may be carried out at most 600 minutes, at most 540 minutes, at most 480 minutes, at most 420 minutes, at most 360 minutes, at most 300 minutes, at most 240 minutes, at most 180 minutes, at most 120 minutes, at most 60 minutes, or at most 30 minutes.

The hydrothermal reaction carried out in phenolics conversion unit 16 can provide for conversion of lignin-derived phenolics into desirable hydrocarbons that can be used in a fuel blends, such as gasoline. Illustrative, non-limiting hydrocarbon compounds include alkanes, alkenes, cycloalkanes and their alkyl substituents or derivatives, and cycloalkenes and their alkyl substituents or derivatives, which can be suitable for use in fuel composition, for instance gasoline or diesel. For instance, illustrative hydrocarbon compounds can include but are not limited to any one of cyclohexane, cyclohexene, propyl cyclopentane, propyl cyclopentene, propyl cyclohexane, propyl cyclohexene, anisole, propyl benzene, oxygenated hydrocarbons cyclohexanone or methyl cyclohexanone, and methyl propyl benzene. The conversion of lignin derived phenolics into desirable hydrocarbons may not be complete, which can leave unconverted phenolics still remaining in the second reaction product. Optionally, the hydrothermal reaction carried out in phenolics conversion unit 16 can also provide for hydrodeoxygenation where triols and glycol of the alcoholic component are converted to monohydric alcohol. The hydrodeoxygenation may not be complete, which can leave triols and glycol still remaining in the second reaction product. As such, the second reaction product can comprise unconverted phenolics, hydrocarbons converted from phenolics, and at least a portion of the alcoholic component.

In phenolics conversion unit 16, phenols can also be converted to a cyclic alcohol, including at least one of alkyl cyclohexanols and alkyl cyclopentanols, which can similarly serve as a portion of the extraction solvent. Operating conditions of phenolics conversion unit 16 can be selected to generate relatively more of certain phenols-derived cyclic alcohols, thereby leaving less phenols in the second reaction content in unit 16. At least one of cyclohexanol and cyclopentanol can also be formed from carbohydrates in hydrothermal digestion unit 2 and phenolics conversion unit 16, if present, via hydrodeoxygenation reactions in the presence of a catalyst capable of activating molecular hydrogen.

As mentioned, it has been found that a second hydrothermal reaction step, which takes in reaction zone 17, such as in phenolics conversion unit 16 of FIG. 2, with a low phenolics concentration provides better yields of hydrocarbons than one with a high concentration of phenolics. Phenolics concentration in unit 16 includes a concentration of up to 50% by weight based on the total weight of the content of phenolics conversion unit 16. Non-limiting illustrative phenolics concentrations of the reaction content of unit 16 can be in a range of about 0.1% to 50% by weight, and any amount in between, including less than 45%, less than 40%, less than 35%, less than 30%, less than 25%, less than 20%, less than 15%, less than 10%, or less than 5% by weight, based on the total content weight of the second hydrothermal reaction step.

Also mentioned was that conversion of lignin to phenols and/or conversion of phenolics to hydrocarbon compounds can be improved if the conversion is accomplished in the presence of water. Referring to FIG. 2, phenolics concentration in unit 16 of 50% or less by weight of the content in unit 16 can be achieved at least in part with water. For instance, the concentration of water in phenolics conversion unit 16 can be at least 10% by weight based on the total weight of the content of unit 16. Non-limiting illustrative water concentration of the reaction content in phenolics conversion unit 16 can be at least 10%, at least 15%, at least 20%, at least 25%, at least 30%, at least 35%, or at least 40% by weight, based on the total content weight of the second hydrothermal reaction step.

FIGS. 1-2 show acid addition to the process stream via 71A, 71B or 71C. By addition of an inorganic acid, it helps to revert some carboxylate salts to form free organic acids and thereby prevent loss of yield. The carboxylates salts can be any of C1-C6 carboxylic acids salts such as formates, acetates, lactates, propionate, and/or levulinates salts. The acids may be any inorganic acids such as sulfuric acid, nitric acid, phosphoric acid, hydrochloric acid and the like. For example, by addition of hydrochloric acid, acetate salt (such as sodium acetate) may be converted to acetic acid that may be taken by overhead to further processing unit to be reacted to produce higher hydrocarbon products and form sodium chloride. The acid addition should be conducted after the digestion reactions (10 and 16) but before the aqueous separation zone 25.

FIGS. 1-2 show gas separator unit 18, which is optional. In FIG. 1, the first reaction product is provided to gas separator unit 18 via line 19. In FIG. 2, the second reaction product is provided to gas separator unit 18 via line 19. Gas separator unit 18 allows for removal of excess gas in the reaction product, as desired or needed. As discussed above, the first reaction product from hydrothermal thermal digestion unit 2 and the second reaction product from phenolics conversion unit 16 are provided to gas separator unit 18 under pressure because the respective unit 2 and unit 16 are operated in a pressurized state. As such, excess gas can be released from the reaction product by keeping gas separator unit 18 under the same pressure as or reduced pressure compared to hydrothermal digestion unit for system 1A in FIG. 1, or under the same pressure as or reduced pressure compared to phenolics conversion unit 16 for system 1B in FIG. 2. As shown in FIG. 1-2, the excess gas can be provided to further processing zone 22 via line 23. A major portion of the excess gas comprises hydrogen and other compounds with a normal boiling point of less than 100 degrees C., including methane, carbon dioxide, carbon monoxide, and water). Other compounds, such as light mono-oxygenates, particularly less than C4 may be present in the excess gas, where this stream may provide addition material for conversion to a fuels product in further processing zone 58. The pressure of gas separator unit 18 can be selected based on considerations of the desired or selected operating pressure of liquid-liquid separation unit or liquid-liquid extraction unit 20 in separation zone 13 and/or the desired or selected operating pressure of further processing zone 58. For instance, one consideration to not depressurize the reaction product in gas separator unit 18 below a certain amount is the need to repressurize products coming out of separation zone 13 intended for further processing zone 58. As such, it is understood that the pressure of gas separator unit 18 may be dependent on the pressure of other units and/or zones in system 1A or 1B. Non-limiting illustrative examples of a pressure of gas separator unit 18 can be in a range of about 5 to 50 bar, including 10 bar, 15 bar, 20 bar, 25 bar, 30 bar, 35 bar, 40 bar, or 45 bar.

As mentioned, gas separator unit 18 is optional so the first reaction product or the second reaction product can be provided to separation zone 13 from hydrothermal digestion unit 2 or phenolics conversion unit 16, respectively, without going through gas separator unit 18. Referring to FIGS. 1-2, separation zone 13 comprises liquid-liquid separation or liquid-liquid extraction unit 20, aqueous separation zone 25, non-aqueous distillation unit 27, and optionally lignin depolymerization unit 29. In liquid-liquid separation or liquid-liquid extraction unit 20, the reaction product provided via line 26 is separated into an aqueous stream depicted as line 28 and a non-aqueous depicted as line 24. The aqueous stream in line 28 is provided to aqueous separation zone 25. The non-aqueous stream in line 24 is provided to non-aqueous distillation unit 27. With lignin depolymerization unit 29, the non-aqueous stream with depolymerized lignin can be provided via line 30 to optional lignin precipitation zone 62.

Liquid-Liquid phase separation unit or Liquid-liquid extraction unit 20 allows for separation and/or extraction of various compounds from the aqueous phase of the reaction product by an extraction solvent. This may be achieved by using an extraction solvent in which the compounds targeted for extraction have a higher solubility than in the aqueous phase. At least a portion of the extraction solvent may be provided from the reaction product, that can be a high hydrocarbon product (which may also be a distillation fraction thereof that is collectively referred to as “higher hydrocarbon products”) 49, a middle fraction from the distillation unit or even a bottom fraction of a non-aqueous stream flasher (not shown) as described herein. As shown in FIGS. 1-2, an extraction solvent may be provided via line 45. As shown, the solvent in line 45 may be optionally combined, via line 45A, with the reaction product in line 26 before entering liquid-liquid extraction unit 20. Additionally or alternatively, the solvent in line 45 may be provided to unit 20 via line 45B for mixing with the reaction product of line 26 in unit 20.

As shown in FIGS. 1-2, the higher hydrocarbon stream for lignin precipitation may come from further processing zone 58 by line 53. Further optionally at least a portion of the extraction solvent in line 45 may come from further processing zone 58 by line 49. At least a portion of the higher hydrocarbon stream for lignin precipitation and optionally for extraction solvent can also be provided from the further processing unit 22 or from an optional distillation unit 50. The lignin precipitation and optionally liquid-liquid extraction degree can be optimized as desired depending on the fraction 51 that is recycled to the respective zones. In one embodiment such higher hydrocarbon fraction 51 may be an aromatics-rich hydrocarbon fraction. In another embodiment, the higher hydrocarbon fraction 51 may be a lower-aromatics hydrocarbon fraction. In another embodiment, it may be a combination of such streams or another any fractionation 51 of the higher hydrocarbon products from further processing unit. 52 is an optional higher hydrocarbon fraction such as moderate fraction if 51 is a light fraction, or 52 is a light fraction if 51 is a moderate fraction. 54 is a heaviest fraction.

If desired, at least a portion of the lignin may be removed or separated in the lignin separation unit 60 as described above. Such treated stream is then provided with or without separation to the distillation unit 27 via line 31. If the lignin is not separated in a lignin separation unit 60, the lignin may be separated more readily in the distillation unit 27.

If present, the extraction solvent in line 45 can be provided at a ratio in a range of 1 part solvent in line 45 to 9 parts reaction product in line 26 to 9 parts solvent in line 45 to 1 part reaction product in line 26. Non-limiting illustrative ratios include 1 part solvent to 9 parts reaction product (1:9), 1 part solvent to 8 parts reaction product (1:8), 1 part solvent to 7 parts reaction product (1:7), 1 part solvent to 6 parts reaction product (1:6), 1 part solvent to 5 parts reaction product (1:5), 1 part solvent to 4 parts reaction product (1:4), 1 part solvent to 3 parts reaction product (1:3), 1 part solvent to 2 parts reaction product (1:2), 1 part solvent to 1 part reaction product (1:1), 2 parts solvent to 1 part reaction product (2:1), 3 parts solvent to 1 part reaction product (3:1), 4 parts solvent to 1 part reaction product (4:1), 5 parts phenols to 1 part reaction product (5:1), 6 parts solvent to 1 part reaction product (6:1), 7 parts solvent to 1 part reaction product (7:1), 8 parts solvent to 1 part reaction product (8:1), 9 parts solvent to 1 part reaction product (9:1), 10 parts solvent to 1 part reaction product (10:1), and any combination thereof.

The reaction product generated by either hydrothermal digester 2 in FIG. 1 or phenolics conversion unit 16 in FIG. 2 is a mixture of an aqueous phase and at least one non-aqueous phase, so it is typically an emulsion that tends to have a higher amount of water than other compounds. When the amount of extraction solvent in liquid-liquid extraction unit 20 is in the lower end, the liquid-liquid extraction process may behave more like liquid-liquid phase separation where the extraction solvent helps break the reaction product emulsion into layers and/or phases by increasing the concentration of the non-aqueous phase(s) to bring it closer to a 1:1 ratio with the aqueous phase. In the lower end, the level of extraction of components from the aqueous phase into the solvent is minimal. The material in line 45 is considered an extraction solvent because it has a different composition from the non-aqueous liquid phase being separated from the reaction product in line 26. As the amount of extraction solvent added to liquid-liquid extraction unit 20 is increased, the level or extent of extraction of certain compounds also increases. The conditions of liquid-liquid extraction unit 20 can be selected to maximize the level of extraction.

Referring to FIGS. 1-2, any suitable liquid-liquid extraction methods and systems capable of partitioning the reaction product into the aqueous stream and the non-aqueous stream can be used. These methods can be operated in batch mode, semi-batch mode, continuous mode, or any combination thereof.

Suitable types of apparatus that can be employed in the liquid-liquid extraction include stage-type extractors or differential extractors. An example of a stage-type extractor is a mixer-settler apparatus that typically includes a mixing tank coupled to a settling vessel to allow the phases to coalesce. In general, a mixer-settler apparatus can be used in batch mode, or a plurality of mixer-settler apparatus can be staged to operate in a continuous or semi-continuous mode. Another stage-type extractor is a centrifugal contactor. Various types of differential extractors (also known as “continuous contact extractors,”) that are also suitable include, but are not limited to, centrifugal contactors and contacting columns such as tray columns, spray columns, packed towers, rotating disc contactors and pulse columns.

In general, a mixer-settler apparatus useful in liquid-liquid extraction unit 20 includes a mixer vessel with an agitator to provide mixing of the solvent provided by line 45 and reaction product provided by line 26. The mixed contents can be provided to a settling tank where the phases can coalesce and separate so an aqueous stream can be withdrawn via line 28 and a non-aqueous stream can be withdrawn via line 24.

In general, centrifugal contactors are high-speed, rotary machines characterized by relatively low residence time. The number of stages in a centrifugal device is usually one, however, centrifugal contactors with multiple stages can also be used. Centrifugal contactors utilize mechanical devices to agitate the mixture to increase the interfacial area and decrease the mass transfer resistance. Various types of differential extractors (also known as “continuous contact extractors,”) that are also suitable for use as unit 20 include, but are not limited to, centrifugal contactors and contacting columns such as tray columns, spray columns, packed towers, rotating disc contactors and pulse columns.

Contacting columns are suitable for various liquid-liquid extraction operations. Packing, trays, spray or other droplet-formation mechanisms or other apparatus are used to increase the surface area in which the two liquid phases (i.e., a solvent phase and a hydrocarbon phase) contact, which also increases the effective length of the flow path. Changes in flows and physical properties along the length of an extractor can also be considered in selecting the type of extractor and/or the specific configuration, materials or construction, and packing material type and characteristics (i.e., average particle size, shape, density, surface area, and the like). For instance, the extraction solvent in line 45 may be provided in unit 20 in a counter-current flow direction relative to the flow of reaction product in line 26.

In particular, liquid-liquid separation unit 20 may include a tray column where the reaction product in line 26 is provided to the bottom of the column and the extraction solvent is provided at or near the top of the column. In addition, the column can be a multi-stage counter-current contractor having a plurality of trays and associated down comers as known to one of ordinary skill.

An additional type of unit operation suitable as liquid-liquid extraction unit 20 is a packed bed column, which typically comprises suitable packing material including, but not limited to, Pall rings, Raschig rings, Kascade rings, Intalox saddles, Berl saddles, super Intalox saddles, super Berl saddles, Demister pads, mist eliminators, telerrettes, carbon graphite random packing, other types of saddles, and the like, including combinations of one or more of these packing materials. Packing material provides large interfacial areas for phase contacting, causing the droplets to coalesce and reform.

Further types of apparatus suitable for liquid-liquid extraction include rotating disc contactors. The rotating disc contactor is a mechanically agitated, counter-current extractor. Agitation is provided by a rotating disc mechanism.

An additional type of apparatus suitable for aromatic extraction in the system and method of the present invention is a pulse column, which has a plurality of packing or sieve plates lacking down comers. The perforations in the sieve plates typically are smaller than those of non-pulsating columns. A pulse-producing device, such as a reciprocating pump, pulses the contents of the column at frequent intervals. The rapid reciprocating motion, of relatively small amplitude, is superimposed on the usual flow of the liquid phases. The pulsation causes the contact between the phases to affect extraction.

The liquid-liquid extraction process can also involve apparatus that allows or facilitates coalescing of the phases, including a horizontal settling tank, one or more weirs, centrifuge- or hydroclone-type device, a fiber coalesce mat or a packed bed coalescer. As mentioned, liquid-liquid extraction unit 20 provides aqueous stream in line 28 and non-aqueous stream in line 24.

The aqueous stream comprises a major portion of the water present in the reaction product in liquid-liquid extraction unit 20. For instance, the aqueous stream comprises greater than 50 wt %, including 55 wt % or greater, 60 wt % or greater, 70 wt % or greater, 75 wt % or greater, 80 wt % or greater, 85 wt % or greater, 90 wt % or greater, and 95 wt % or greater of the water in the reaction product in unit 20. Correspondingly, the non-aqueous stream comprises a minor portion of the water in the respective reaction product in liquid-liquid extraction unit 20. For instance, the non-aqueous stream comprises up to 50 wt %, including an amount in a range of 0.1 wt % and up to 50 wt %, up to 45 wt %, up to 40 wt %, up to 35 wt %, up to 30 wt %, up to 25 wt %, up to 20 wt %, up to 15 wt %, up to 10 wt %, or up to 5 wt % of water in the reaction product in unit 20. The non-aqueous stream comprises the remaining portion of the reaction product not in the aqueous stream. For instance, the non-aqueous stream may have more than one phase.

If the relative amount of extraction solvent employed is low, such as when the extraction solvent is provided at less than 50% by weight relative to the reaction product in line 26, the extent of the extraction may be minimal so the aqueous stream can also comprise a major portion of compounds at least partially miscible with water in the reaction product line 26 and provided to unit 20, including light compounds, middle-boiling compounds, heavy compounds, in addition to water-soluble salts, and ash. The light compounds include those that have a normal boiling point of less than 150 degrees C. Non-limiting illustrative examples of light compounds include monohydric alcohols, aldehydes, ketones, acids, and any combination thereof. The middle-boiling compounds present in the aqueous stream include those that have a normal boiling point in a range of 150 to 350 degrees C. or greater, which can include glycols and phenols. The heavy compounds include those that have a normal boiling point of greater than 350 degrees C.

If the relative amount of extraction solvent provided is high, such as when the extraction solvent is provided at greater than 50% by weight relative to the reaction product in line 26, the aqueous stream may be largely depleted of light compounds targeted for extraction, including at least one of mono-oxygenates (such as C1-C4), certain diols, and free acids (such as acetic acid or higher). That is, in these circumstances, the aqueous stream may contain a minor portion of light compounds targeted for extraction in the reaction product in line 26 and provided to unit 20, where these light compounds include at least one of mono-oxygenates (such as C1-C4), certain diols, and free acids (such as acetic acid or higher). In these circumstances, the aqueous stream may still comprise glycols, including ethylene glycol and propylene glycol, as well as some amount of the extraction solvent. The aqueous stream also contains water soluble salts, including acid salts and ash.

Referring to FIGS. 1-2, the aqueous stream can be provided via line 28 to aqueous separation zone 25. After the liquid-liquid extraction process, if the aqueous stream contains a threshold amount of light compounds considered to be recoverable by one of ordinary skill, such as when the level of extraction is on the lower end, the light compounds can be recovered in aqueous separation zone 25. The aqueous stream can be subject to flashing with reduced steam stripping effect because the aqueous stream contains a reduced amount of phenols compared to the reaction product having multiple liquid phases, such that a separate phenolic-rich phase can be minimized during the flashing step Flashing of the aqueous stream recovers various light compounds that can be further processed into a fuel product. Light compounds can include those with a normal boiling point of less than 150 degrees C., including but not limited to monohydric alcohols, aldehydes, ketones, acids, and any combination thereof.

Referring to FIGS. 1-2, a major portion of light compounds in the aqueous stream is recovered in aqueous separation zone 25. For instance, more than 50 wt %, particularly at least 75 wt %, at least 80 wt %, at least 85 wt %, at least 90 wt %, or at least 95 wt % of compounds with a normal boiling point of less than 150 degrees C. in the aqueous stream in zone 25 is recovered. These light compounds can be provided via line 32 to further processing zone 58 where they can be further processed in a desired product, as further discussed below. The light compounds coming from aqueous separation zone 25 via line 32 may be combined with excess gas in line 23 from gas separator unit 18, if present, before entering further processing zone 58 as shown in FIGS. 1-2. Additionally or alternatively, it may enter zone 58 separately. In certain instances, heavier compounds, such as those with a normal boiling point of 150 degrees C. or greater, including phenols and glycols, may also be recovered in aqueous separation zone 25. If these heavier compounds are recovered in zone 25, they can be provided to digestion zone 3 and/or reaction zone 17 via line 33 as shown in FIGS. 1-2. In particular, FIG. 1 show line 33 between hydrothermal digestion unit 2 and aqueous separation zone 25, and FIG. 2 show line 33 between phenolics conversion unit 16 and zone 25. It is understood that line 33 can run to hydrothermal digestion unit 2 in FIG. 2 even though it is not shown. As mentioned, glycol and/or phenols can be used as part of the digestion solvent. Contaminants such as salts, ash, caramel, and tar remaining can be disposed of as waste via line 34.

If the relative amount of extraction solvent supplied to unit 20 is high, such as greater than 1 part extraction solvent to 2 parts reaction product in line 26, recovery of light compounds in the aqueous stream in line 28 may not be necessary or desirable. As such, the aqueous stream may not be processed in aqueous separation zone 25 as described, thereby rendering zone 25 optional. Additionally or alternatively, the handling of the aqueous stream in zone 25 may be configured to provide the heavier compounds like phenols and glycols instead of the light compounds, thereby rendering line 32 optional.

Aqueous separation zone 25 can comprise any suitable method known to one of ordinary skill in the art used to separate these light compounds. For instance, zone 25 can comprise one or more flashers. In one circumstance, which is not shown, zone 25 can comprise one flasher to flash the aqueous stream to recover light compounds in the overhead fraction while the bottom fraction containing water-soluble salts and ash can be discarded as waste. The bottom fraction from a single flash, however, can also contain other valuable heavier compounds, such as phenols and glycols. FIG. 3 shows one option of aqueous separation zone 25 to recover these valuable heavier compounds where zone 25 comprises more than one flasher: flasher 35 and flasher 36. In FIG. 3, light compounds are recovered in the overhead fraction of flasher 35, which is provided to further processing zone 58 via line 32. The bottom fraction of flasher 35 is provided to flasher 36 via line 37. In flasher 36, heavier valuable compounds, including phenols and glycols, are recovered in the overhead fraction, which can be provided to digestion zone 3 and/or reaction zone 17 via line 33. Contaminants exit as waste via line 34. Other suitable separation methods that may be employed in aqueous separation zone 25 includes use of membranes for separation of organics and salts from water.

Referring to FIGS. 1-2, as mentioned, the non-aqueous stream coming from liquid-liquid extraction unit 20 can be provided to optional lignin depolymerization unit 29 before entering non-aqueous distillation unit 27. If present, the non-aqueous stream can enter unit 29 via line 24. Lignin depolymerization unit 29 can improve product separation of the non-aqueous stream by reducing its viscosity through one or more lignin depolymerization reactions. Lignin depolymerization unit 29 can comprise any suitable lignin depolymerization reaction.

Suitable lignin depolymerization reactions are known in the art. Non-limiting illustrative lignin depolymerization reactions include thermal reactions, which can be performed with or without catalysts. Generally, thermal lignin depolymerization reactions performed without catalysts typically take place in a temperature of greater than 300 degrees C. Catalysts and hydrogen can be added to reduce the reaction to below 300 degrees C. Any suitable catalyst can be employed, including acid or basic catalysts, in solid or liquid form. For example, methods relating to lignin depolymerization catalysts using a solid catalyst are described in U.S. Application Publication No. 2012/0302796, the disclosures of which are incorporated by reference in its entirety. Other disclosures relating to using catalysts in lignin depolymerization include U.S. Pat. Nos. 6,100,385 and 7,964,761, the disclosures of which are incorporated by reference in their entirety. Other methods to depolymerize lignin in the non-aqueous stream include pyrolysis (thermolysis), gasification, hydrogenolysis, chemical oxidation, and hydrolysis under supercritical conditions. Additional details regarding these methods can be found in Pandey & Kim, “Lignin Depolymerization and Conversion: A Review of Thermochemical Methods,” Chem. Eng. Technol., 2011, No. 1, 29-41, the disclosure of which is incorporated by reference in its entirety. If lignin is still present in the non-aqueous stream prior to full distillation, it may become precipitated and form a slurry of lignin. In this circumstance, optionally, a lignin removal mechanism may be used to capture at least a portion of this lignin in the non-aqueous stream prior to full distillation. Any suitable lignin removal mechanism may be used. Non-limiting suitable lignin removal mechanism includes a filter, a press, a hydroclone apparatus, or any combination thereof. The lignin captured by the lignin removal mechanism may be provided to lignin depolymerization unit 29 for further depolymerization or any other depolymerization step.

Referring to FIGS. 1-2, the non-aqueous stream with optionally depolymerized lignin is provided to non-aqueous distillation unit 27, which can comprise any suitable distillation systems or methods, including multi-stage distillation. As shown, in non-aqueous distillation unit 27, the non-aqueous stream is distilled into various product fractions, including an overhead fraction, one or more middle fractions, and a bottom fraction.

An overhead fraction separated from the non-aqueous stream, which is depicted as line 38, includes a major portion of light compounds in the non-aqueous stream provided to distillation unit 27 via line 30. For instance, more than 50 wt %, particularly at least 75 wt %, at least 80 wt %, at least 85 wt %, at least 90 wt %, or at least 95 wt % of compounds with a normal boiling point of less than 150 degrees C. present in the non-aqueous stream in distillation unit 27 is in the overhead fraction in line 38.

The middle fraction can comprise compounds heavier than the light compounds, particularly those with a normal boiling point of 150 degrees C. or greater. Distillation unit 27 can be operated to provide more than one middle fraction. For instance, distillation can be operated to provide a first middle fraction, depicted as line 39, with a normal boiling point of about 150 to 250 degrees C., which typically captures phenolic compounds, and a second middle fraction, depicted as line 40, with a higher normal boiling point of 200 to 350 degrees C., which typically captures lignin oligomers. Distillation can also be operated to provide a middle fraction with a normal boiling point of about 150 to 350 degrees C. As mentioned above, there is an overlap in the normal boiling point of the middle fractions, which is at least due to the distribution of compounds during the distillation process. Accordingly, the first middle fraction in line 39 can include compounds with a normal boiling point in a range of about 150 to 300 degrees C. The second middle fraction, which is depicted as line 40, can include compounds with a normal boiling point of 200 to 350 degrees C., which typically captures lignin oligomers. The bottom fraction depicted as line 41 has compounds with a normal boiling point of greater than 350 degrees C., which typically capture the heaviest of compounds regarded as waste or byproducts, such as caramel and/or tar. While the first middle fraction may be referred to as the phenols fraction and the second middle fraction may be referred to as the lignin oligomers fraction, it is understood that the phenols fraction may contain lignin oligomers, and the lignin oligomers may contain phenols. It is understood that a middle fraction distilled from the non-aqueous stream can be the phenols fraction, the lignin oligomers fraction, or a combination thereof. As described, separation zone 13 provides one or more middle fraction that contains a major portion of phenolics in the reaction product provided to separation zone 13.

Referring to FIGS. 1-2, the overhead fraction from the distillation unit 27 can be provided to further processing zone 58 via line 38. As shown, material in line 38 may be combined with other light compounds in line 32 recovered from the aqueous stream and/or excess gas in line 23 recovered from gas separator unit 18, if present, before entering further processing unit 22. Additionally or alternatively, material in line 38 may be provided to further processing zone 58 separately.

Referring to FIGS. 1-2, at least a portion of the one or more middle fractions from distillation unit 27 can be recycled to various locations in system 1. For the sake of simplicity, the lines connecting a middle fraction from distillation unit 27 to other places in system 1 have not been shown. Instead, inlets and partial depictions of the lines are provided. It is understood that these lines may be connected to each other as described even if the full depiction is not provided. Referring to FIGS. 1-2, one or more middle fractions may also be provided to at least one of digestion zone 2, reaction zone 17, and lignin depolymerization unit 29. In FIGS. 1-2, the first middle fraction in line 39 may be provided to hydrothermal digestion unit 2 via line 39 that is connected to inlet 44 for use as part of the digestion solvent. Additionally or alternatively, while not preferred, the second middle fraction in line 40 may be provided to hydrothermal digestion unit via line 40 that is connected to inlet 44. It is also possible to combine the first middle fraction in line 39 with the second middle fraction in line 40, via lines 39A and 40A, where the mixture of both can be provided to hydrothermal digestion unit 2 via line 46 that is connected to inlet 44. A single middle fraction comprising both the first middle fraction and a second middle fraction may be provided to hydrothermal digestion unit 2 as well. The amount of the material in line 39, 40, or 46 provided to hydrothermal digestion unit 2 may be determined by the amount of lignin provided to hydrothermal digestion unit 2 as discussed above.

Additionally or alternatively, in FIG. 2, at least one middle fraction from distillation unit 27 is also provided to phenolics conversion unit 16 via line 42. In FIG. 2, the first middle fraction in line 39 is provided to unit 16 via line 39 that is connected to line 42. Additionally or alternatively, the second middle fraction in line 40 is provided to unit 16 via line 40 that is connected to line 42. Additionally or alternatively, the first middle fraction in line 39 may be combined with the second middle fraction in line 40, via lines 39A and 40A, where the mixture of both can be used as part of an extraction solvent and provided via line 46 that is connected with line 42. A single middle fraction comprising both the first middle fraction and a second middle fraction may be provided to phenolics conversion unit 16 as well. The phenols in at least one of these fractions can be further converted into hydrocarbons in unit 16 as described herein. The lignin in at least one of these fractions can be further reverted into phenols in unit 16 as described herein.

Referring to FIG. 2, the amount of a middle fraction distilled from the non-aqueous stream provided to conversion unit 16 affects the amount of phenolics concentration in unit 16. As mentioned, it is preferred that the phenolics concentration, which includes the amount of lignin oligomers, in phenolics conversion unit 16 is maintained at 50 wt % or below. If the first reaction product from hydrothermal digestion unit 2 that goes to phenolics conversion unit 16 already contains a certain phenolics concentration, this is taken into account if a portion of a middle fraction distilled from the non-aqueous stream is added to unit 16 so that the phenolics concentration in unit 16 can be maintained as needed or desired. For instance, the fraction from at least one of line 39 and line 40 may be provided to phenolics conversion unit 16 at a ratio to or percentage the first reaction product in line 12. In particular, a non-limiting, illustrative example includes the amount of material from at least one of line 39 and line 40 is no more than 50 wt % of the amount or flow rate of the first reaction product in line 12. In this circumstance, the amount of the first reaction product is at least two parts to one part of material from at least one of line 39 and line 40.

As shown in FIGS. 1-2, material from at least one of line 39 and line 40 can also be provided to lignin depolymerization unit 29, if present, via line 43. At least the lignin in the material from at least one of line 39 and line 40 can be further depolymerized, which improves distillation efficiency in distillation unit 27, as the viscosity of the material provided via line 30 is further reduced.

Some or all of material in at least one of line 39 and line 40 may be provided to lignin depolymerization unit 29. For instance, at least 10 wt % of the material in a middle fraction distilled from the non-aqueous stream is provided to lignin depolymerization unit 29, particularly, at least 20 wt %, at least 30 wt %, at least 40 wt %, at least 50 wt %, at least 60 wt %, at least 70 wt %, at least 80 wt %, or at least 90 wt %. The amount may be selected by one of ordinary skill in the art by balancing different factors, including the use of phenols as solvent in liquid-liquid extraction unit 20, hydrothermal digestion unit 2, and further lignin reversion and conversion to hydrocarbons in phenolics conversion unit 16. Recycling more material to one location leaves less material for recycling to other locations. An external solvent (i.e., one that is not recycled) may be provided to system 1, particularly in hydrothermal digestion unit 2, at start up or as needed.

Referring to FIGS. 1-2, further processing unit 22 can comprise any suitable number of reactors coupled to one another, such as at least one, two, three, four, five, or six further processing units. The reaction(s) taking place in further processing zone 58 can convert the alcoholic component to the desired hydrocarbon compounds. While not shown, hydrocarbons converted from phenolics that are in the second reaction product may be separated from the second reaction product before it is routed to further processing zone 58, or the hydrocarbons may remain in the second reaction product and go through further processing zone 58 as described herein. Also, hydrogen and water vapor may or may not be removed prior to the further processing of the remaining fraction.

Referring to FIGS. 1-2, further processing zone 58 may generally comprise a condensation reaction, often conducted in the presence of a condensation catalyst, in which the alcoholic component or a product formed therefrom is condensed with another molecule to form a higher molecular weight compound. As used herein, the term “condensation reaction” will refer to a chemical transformation in which two or more molecules are coupled with one another to form a carbon-carbon bond in a higher molecular weight compound, usually accompanied by the loss of a small molecule such as water or an alcohol. An illustrative condensation reaction is the Aldol condensation reaction, which will be familiar to one having ordinary skill in the art.

Although a number of different types of catalysts may be used for mediating condensation reactions, zeolite catalysts also may be particularly advantageous in this regard. One zeolite catalyst that may be particularly well suited for mediating condensation reactions of alcohols is ZSM-5 (Zeolite Socony Mobil 5), a pentasil aluminosilicate zeolite having a composition of NanAlnSi96-nO192.16H₂O (0<n<27), which may transform an alcohol feed into a condensation product. Other suitable zeolite catalysts may include, for example, ZSM-12, ZSM-22, ZSM-23, SAPO-11, and SAPO-41.

In various embodiments, the condensation reaction may take place at a temperature ranging between about 275 degrees C. and about 450 degrees C. The condensation reaction may take place in a condensed phase (e.g., a liquor phase) or in a vapor phase. For condensation reactions taking place in a vapor phase, the temperature may range between about 300 degrees C. and about 400 degrees C., such as 350 degrees C. or above. The condensation reaction may take place at a pressure in a range of about 5 bar to 50 bar, such as 10 bar to 30 bar, including about 15 bar to 20 bar.

The alcoholic component, particularly when it includes methanol and an oxygenate, such as such as at least one of ketones, aldehydes, furans, and ethers, can provide for improved conversion of methanol over to a fuel compound, such as gasoline or diesel, as compared to other conventional methanol conversion processes. An example of such a conventional methanol conversion process is the methanol-to-gasoline process.

The higher molecular weight compound produced by the condensation reaction may comprise >C4 hydrocarbons, such as C4-C30 hydrocarbons, C4-C24 hydrocarbons, C4-C18 hydrocarbons, or C4-C12 hydrocarbons; or >C6 hydrocarbons, such as C6-C30 hydrocarbons, C6-C24 hydrocarbons, C6-C18 hydrocarbons, or C6-C12 hydrocarbons. As used herein, the term “hydrocarbons” refers to compounds containing both carbon and hydrogen without reference to other elements that may be present. Thus, heteroatom-substituted compounds are also described herein by the term “hydrocarbons.” The particular composition of the higher molecular weight compound produced by the condensation reaction may vary depending on the catalyst(s) and temperatures used for both the catalytic reduction reaction and the condensation reaction, as well as other parameters such as pressure.

A single catalyst may mediate the transformation of the alcoholic component into a form suitable for undergoing a condensation reaction as well as mediating the condensation reaction itself. Zeolite catalysts are one type of catalyst suitable for directly converting alcohols to condensation products in such a manner. A particularly suitable zeolite catalyst in this regard may be ZSM-5, although other zeolite catalysts may also be suitable.

On the other hand, a first catalyst may be used to mediate the transformation of the alcoholic component into a form suitable for undergoing a condensation reaction, and a second catalyst may be used to mediate the condensation reaction. Unless otherwise specified, it is to be understood that reference herein to a condensation reaction and condensation catalyst refers to either type of condensation process. Further disclosure of suitable condensation catalysts now follows. Zeolite catalysts may be used as either the first catalyst or the second catalyst. Again, a particularly suitable zeolite catalyst in this regard may be ZSM-5, although other zeolite catalysts may also be suitable.

Various catalytic processes may be used to form higher molecular weight compounds by a condensation reaction. In some embodiments, the catalyst used for mediating a condensation reaction may comprise a basic site, or both an acidic site and a basic site. Catalysts comprising both an acidic site and a basic site will be referred to herein as multi-functional catalysts. In some or other embodiments, a catalyst used for mediating a condensation reaction may comprise one or more metal atoms. Any of the condensation catalysts may also optionally be disposed on a solid support, if desired. Additional details regarding suitable catalysts are described in commonly owned United States Patent Application Publication US20140117277, and entitled Methods and Systems for Processing Lignin During Hydrothermal Digestion of Cellulosic Biomass Solids,” the entire disclosure of which is incorporated herein by reference.

For example, the condensation catalyst may also include a zeolite and other microporous supports that contain Group IA compounds, such as Li, Na, K, Cs and Rb. Preferably, the Group IA material may be present in an amount less than that required to neutralize the acidic nature of the support. A metal function may also be provided by the addition of group VIIIB metals, or Cu, Ga, In, Zn or Sn. In some embodiments, the condensation catalyst may be derived from the combination of MgO and Al₂O₃ to form a hydrotalcite material. Another condensation catalyst may comprise a combination of MgO and ZrO₂, or a combination of ZnO and Al₂O₃. Each of these materials may also contain an additional metal function provided by copper or a Group VIIIB metal, such as Ni, Pd, Pt, or combinations of the foregoing.

The condensation reaction mediated by the condensation catalyst may be carried out in any reactor of suitable design, including continuous-flow, batch, semi-batch or multi-system reactors, without limitation as to design, size, geometry, flow rates, and the like. The reactor system may also use a fluidized catalytic bed system, a swing bed system, fixed bed system, a moving bed system, or a combination of the above. In some embodiments, bi-phasic (e.g., liquid-liquid) and tri-phasic (e.g., liquid-liquid-solid) reactors may be used to carry out the condensation reaction.

In certain embodiments, a system is provided containing: a first reaction zone comprising a reactor configured to heat cellulosic biomass solids, molecular hydrogen, a hydrocatalytic catalyst, and a digestion solvent to form a first reaction product; and a separation zone comprising: an inlet to receive an acid solution in fluid communication with an outlet of the reactor in the first reaction zone and with the liquid-liquid phase separation unit or a liquid-liquid extraction unit; a liquid-liquid phase separation unit or a liquid-liquid extraction unit having an inlet in fluid communication with an outlet of the reactor in the first reaction zone to receive the first reaction product, wherein the liquid-liquid phase separation unit or the liquid-liquid extraction unit is configured to provide an aqueous stream and a non-aqueous stream, wherein the aqueous phase comprises a major portion of water in said portion of the first reaction product; an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction.

In certain embodiments, a system is provided containing: a first reaction zone comprising a reactor configured heat cellulosic biomass solids, molecular hydrogen, a hydrocatalytic catalyst, and a digestion solvent to form a first reaction product; a second reaction zone comprising a reactor in fluid communication with an outlet of the reactor in the first reaction zone to receive the first reaction product, wherein the reactor in the second reaction zone is configured to heat a second reaction content comprising the first reaction product, molecular hydrogen, and a hydrocatalytic catalyst to form a second reaction product; and a separation zone comprising: an inlet to receive an acid solution in fluid communication with an outlet of the reactor in the second reaction zone and with the liquid-liquid phase separation unit or the liquid-liquid extraction unit; a liquid-liquid phase separation unit or a liquid-liquid extraction unit having an inlet in fluid communication with an outlet of the reactor in the second reaction zone to receive second first reaction product, wherein the liquid-liquid phase separation unit or the liquid-liquid extraction unit is configured to provide an aqueous stream and a non-aqueous stream, wherein the aqueous stream comprises a major portion of water in said portion of the first reaction product; an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction; wherein the distillation unit is in fluid communication with an inlet of the reactor in the first reaction zone to provide at least a portion of the middle fraction.

Any of the system described above, may further contain a gas separator unit having an inlet in fluid communication with the first or second reaction zone, wherein the gas separator unit is configured to remove at least a portion of volatile compounds with a normal boiling point of 100 degrees C. or less than in the first reaction product or the second reaction product, wherein an outlet of the gas separator unit is in fluid communication with the liquid-liquid phase separation unit to provide the reaction product without the removed volatile compounds to the liquid-liquid phase separation unit. The gas separator unit may be located after the acid solution inlet or before the acid solution inlet. The distillation unit may be in fluid communication with an inlet of the reactor in the first reaction zone and/or the second reaction zone to provide at least a portion of the middle fraction. The aqueous separation zone comprises: a first flasher configured to produce a first overhead fraction and a first bottom fraction, wherein the first overhead fraction comprises a major portion of compounds with a normal boiling point of less than 150 degrees C. in said portion of the aqueous stream, and wherein the second overhead fraction comprises compounds with a normal boiling point of 150 degrees C. or higher; wherein the first flasher comprises an outlet in fluid communication with an inlet of a second flasher to provide the first bottom fraction to the second flasher. The inlet of the acid solution is located after the first flasher and before the second flasher. The systems may further contain a lignin depolymerization unit in fluid communication with an outlet of the liquid-liquid phase separation unit or a liquid-liquid extraction unit to receive at least a portion of the non-aqueous stream, wherein the thermal reaction zone is configured to provide at least partial depolymerization of lignin in the non-aqueous stream, wherein an outlet of the lignin depolymerization unit is in fluid communication with the distillation unit to provide the non-aqueous stream with at least partial lignin depolymerization. The systems may further contain a further processing zone in fluid communication with at least one of (i) the separation zone to receive at least a portion of the overhead fraction from the distillation unit, (ii) the aqueous separation zone to receive at least a portion of the first overhead fraction from the aqueous separation zone, and (iii) the gas separator unit to receive at least a portion of the removed volatile compounds, wherein the further processing zone is configured to provide a higher hydrocarbon product. The systems may further contain: a lignin removal mechanism having an inlet in fluid communication with an outlet of the lignin depolymerization unit to receive the non-aqueous stream with at least partial lignin depolymerization; wherein the lignin removal mechanism is configured to remove at least a portion of the lignin in the non-aqueous stream; wherein the lignin removal mechanism has an outlet in fluid communication with the distillation unit to provide the non-aqueous stream from the lignin removal mechanism; and wherein the lignin removal mechanism has an outlet in fluid communication with an inlet of the lignin removal mechanism to recycle the non-aqueous stream. The systems may further contain: a biomass wash unit having an outlet in fluid communication with the acid solution inlet and an outlet in fluid communication with the first reaction zone.

The person skilled in the art will readily understand that, while the invention is illustrated making reference to one or more a specific combinations of features and measures, many of those features and measures are functionally independent from other features and measures such that they can be equally or similarly applied independently in other embodiments or combinations.

To facilitate a better understanding of the present invention, the following examples of preferred embodiments are given. In no way should the following examples be read to limit, or to define, the scope of the invention.

### EXAMPLES

### Example 1

### Acid Formation During Digestion Reaction

A solvent mixture was prepared from 10.0 grams of tetrahydrofuran and 45.0 grams of deionized water. A 100-milliliter Parr 4590 series reactor was charged with the solvent mixture, and 3.07 grams of Raney Cobalt 2724 catalyst (WR Grace) containing residual NaOH base. The reactor was then charged with a nominal 3.04 grams of southern pine mini-chips (10% moisture), of nominal size 3×5×5 mm in dimension, before pressuring with 35 bar of hydrogen, and heating with stirring to 190° C. for 1 hour, followed by heating to 245° C. for 4 hours.

pH after a single cycle of wood addition was 5.63. An additional charge of 3.08 grams of 10% wet wood was added for a second reaction cycle. A pH of 5.59 was measured for the aqueous product at the end of cycle #2.

3.00 grams of wood were added for cycle #3, at the end of which a pH of 4.99 was measured. 0.09 grams of KOH were added to bring the pH to 5.44, before addition of 3.02 grams of 10% wet wood to conduct a 4th reaction cycle.

At the end of cycle 4, a pH of 4.67 was measured. 0.91 grams of KOH were added to bring the pH to 5.43 before conducting a fifth reaction cycle. After cycle 5, a pH of 4.75 was measured. 0.3 grams of KOH were added to bring the pH to 6.53, before conducting a reaction kinetic test with 0.31 grams of methylethylketone, heated for 1 hour under hydrogen at 35 bar at 190° C., followed by 3 hours at 245° C. A final pH of 4.71 was determined.

A sample of liquid phase product was analyzed by ion chromatography for acid anion content. A second sample was analyzed by gas chromatography using a 60-m×0.32 mm ID DB-5 column of 1 μm thickness, with 50:1 split ratio, 2 ml/min helium flow, and column oven at 40° C. for 8 minutes, followed by ramp to 285° C. at 10° C./min, and a hold time of 53.5 minutes. Acetone was used as diluent for GC analysis of the bottom droplet or globule phase The injector temperature was set at 250° C., and the detector temperature was set at 300° C. Gas Chromatographic-Mass Spec (GCMS) was effected using the same protocol.

GC and GC-mass spec results indicated formation of ethylene glycol and 1,2-propylene glycol as intermediate products, along with C1-C3 monooxygenates including methanol, ethanol and 1-propanol. Butanediol, butyrolactone, and methylcyclopentanone were observed. Ion chromatography revealed the presence of glycolate at 8146 ppm, formate at 566 ppm, acetate at 6092 ppm, propionate at 2397 ppm, and butyrate at 746 ppm.

A seventh cycle was conducted by heating the reaction mixture to 270° C. under 35 bar hydrogen for 15 hours. The amount of glycolate and formate was reduce by more than a factor of two. However, acetate increased by 15%, propionate and butyrate doubled in concentration.

This example shows that organic acids are formed during digestion and hydrothermal reaction of biomass in the presence of a catalyst capable of activating molecular hydrogen. Use of high temperature reduction does not fully revert the acids to other hydrocarbon or oxygenated hydrocarbon products.

### Example 2

### Liquid Extraction of Neutralized Aqueous Acid Solution

A model reaction mixture was prepared via addition of 2.52 grams of ethanol, 5.01 grams of 1-propanol, 2.52 grams of ethylene glycol, 5.04 grams of 1,2-propylene glycol, 1.86-grams of cresol to 233.10 grams of deionized water.

1.40 grams of glacial acetic acid (98% purity) were added to 200.07 grams of the mixture to obtain a pH of 2.99. 10.09 grams of 1N KOH were added to neutralize the mixture to pH 5.13, simulating intermediate product from the digestion and hydrodeoxygenation reaction of biomass.

7.05 grams of the resulting mixture were extracted with 7.09 grams of n-octanol. Upper and lower layers were analyzed by anion chromatographic method for acetate anion. 0.525 g/kg of acetate anion were present in the upper octanol extractant layer. 5.947 g/kg remained in then aqueous lower layer.

GC analysis was used to assess the partitioning of model components between upper and lower phase. Partition coefficients, expressed as the weight percent present in the upper octanol (oil) rich layer, to the lower aqueous layer, were 0.56 for methanol, 1.99 for propanol, less than 0.01 for ethylene glycol, and 0.03 for propylene glycol. Cresol concentrations in the lower layer were too small to observe, from which a partition coefficient of greater than 100 was estimated.

This experiment demonstrates that extraction of an aqueous reaction mixture from the digestion and HDO reaction of biomass at a pH between 5-7, results in removal of virtually all the phenolic compounds and sufficient monooxygenates to allow recovery by flash distillation and recycle of solvent. Only a small fraction of diols are extracted. The amount of acetate anion recovered by extraction was small, less than 10% of the amount present in the simulated reaction mixture.

### Example 3

### Re-Acidification Via Soluble Acid and Extraction

50-grams of the neutralized model digester intermediate product were acidified by adding 3.79 grams of 1N hydrochloric acid, with mixing. 7.00 grams were extracted with 7.07 grams of n-octanol with vigorous shaking, followed by overnight phase separation.

The concentration of acetate anion observed in the upper layer octanol extract was 1650 ppm, relative to 3767 ppm remaining in the aqueous layer. GC analysis of upper and lower layers revealed little change in the composition of other components.

This result shows an ability to extract free organic acid after reacidifying from basic solution using soluble acid. Separation of acid from octanol extractant followed by recycle of octanol can provide a basis for recovery of acid for condensation to biofuels product.

### Example 4

### Re-Acidification Via Treatment with Acidic Ion Exchange Resin

50.012 grams of the neutralized model digester intermediate product were acidified by contacting with 5.09 grams (wet) of strong acid Amberlyst 15 ion exchange resin, by shaking for 3 hours. A final pH of 2.98 was measured. 7.05 grams of the treated solution were extracted with 7.13 grams of n-octanol with vigorous shaking, followed by overnight phase separation.

The concentration of acetate anion observed in the upper layer octanol extract was 1781 ppm, relative to 3684 ppm remaining in the aqueous layer. GC analysis of upper and lower layers revealed little change in the composition of other components.

This result shows an ability to extract free organic acid after reacidifying from basic solution using ion exchange resin. Separation of acid from octanol extractant followed by recycle of octanol can provide a basis for recovery of acid for condensation to biofuels product.

### Example 5-7

### Distillation of Basic and Re-Acidified Organic Acid Solutions

Approximately 25-gram aliquots of the basic (Example 2) or re-acidified (Examples 3 and 4) aqueous solutions were place in a 50-ml distillation flask fitted with a short path distillation head. Distillations were conducted first under nitrogen with ramping of bottoms temperature to about 150° C., before switching to vacuum at a pressure of about 100 Torr to remove a vacuum cut. Distillation of the aqueous fraction yielded overhead cuts of light monooxygenates (alcohols less than C4), water, and upon increase in bottoms temperature, ethylene glycol and propylene glycol. No detectable phenolics were observed in the overhead cuts from the aqueous phase distillation.

Distillation of the mildly basic pH 5.1 solution from example 2 gave a final bottoms sample of 2.18 grams, or less than 10% of the starting mixture. Acetate concentration in the final bottoms was measured by ion chromatography as 5.1 wt %. 287 ppm acetate appeared in the first distillation cut at atmospheric pressure. 1392 ppm acetate was measured for the average of vacuum distillation cuts.

Example 6 entailed distillation of the soluble hydrochloric acid re-acidified mixture from Example 3. Distillation was stopped after collection of 22.99 grams of overhead product, with a bottoms weight of 2.08 grams. The acetate concentration in the bottoms was only 1.1 wt %, or only about ⅕ of the amount left in the bottoms residue after distillation in Example 5 of the mixture that had not been re-acidified. This example shows that re-acidification to a pH of less than about 4, allows recovery of a substantial amount of organic acid coproduct from hydrothermal digestion and catalyst hydrodeoxygenation of biomass. GC analysis of overheaded products also showed the presence of methanol, ethanol, and propanol for the atmospheric distillation cuts, along with 1125 ppm acetate anion. The vacuum distillation cut included ethylene and propylene glycols, as well as 4200 ppm acetate anion.

Example 7 entailed distillation of the acidic ion exchange resin treated mixture from Example 4, where the pH 5.1 solution was re-acidified to pH 2.98. Distillation was conducted to produce a final bottoms residue of 2.44 grams or less than 10% of the original mixture charged to the distillation flask. Final bottoms contained only 1.01 wt % acetate anion, or less than ⅕ of the amount observed with no-reacidification in Example 5. Overhead cuts containing 544 ppm acetate (atmospheric pressure cut) and 4047 ppm (vacuum cut) were observed.

This example again demonstrates improved recovery of organic acids via distillation following re-acidification to a pH of about 3, vs. distillation of the pH 5-7 product obtained from digestion and hydrodeoxygenation reaction of biomass.

Two methods for recovery of acid coproduct of biomass digestion are thus demonstrated, from a solution which is initially buffered to pH 5-7 for digestion and hydrodeoxygenation reaction. Reacidification to pH of about 3 or less greatly improves the ability to extract organic acids into an organic solvent, from which the acid may be recovered. Alternately, re-acidification of the aqueous solution enables improved recovery of acids as overhead product via flash distillation. Both may be practiced in a process configuration to allow recovery of acidic intermediates, for further processing to biofuels.

### Example 8

### Conversion of Organic Acids to Liquid Fuels

A GC glass capillary injector was packed with 0.07 grams of acidic XSM-5 zeolite catalyst, and heated to 375° C., in front of GC columns operated as described in Example 1, with flame ionization detector, to provide a pulse microreactor assembly for study of biofuels production by acid condensation. A 0.5 microliter aliquot of a mixture of 10% acetic acid in water was injected onto the ZSM-5 pulse microreactor assembly. Peaks of toluene, dimethyl and trimethyl benzenes, with some benzene were observed, together with a small fraction of light alkane products. Product formation could be sustained via multiple injections. Injection of mixtures of sodium acetate led to rapid catalyst deactivation and greatly reduced hydrocarbon product formation.

This example shows that free organic acids can be vaporized and converted to liquid fuels components (gasoline range) via acid condensation reactions.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods may also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

That which is claimed is:

1. A method comprising:
heating cellulosic biomass solids, molecular hydrogen, a catalyst capable of activating molecular hydrogen, and a digestion solvent in a reactor in a first reaction zone to produce a first reaction product;
providing an acid solution to the first reaction product to produce an acidified first reaction product;
providing at least a portion of the acidified first reaction product to a separation zone comprising a liquid-liquid phase separation unit or a liquid-liquid extraction unit to recover an aqueous stream and a non-aqueous stream, wherein the aqueous stream comprises a major portion of water in said portion of the first reaction product;
providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and
providing at least a portion of the non-aqueous stream to a distillation unit to obtain at least an overhead fraction, a middle fraction, and a bottom fraction.

2. The method of claim 1 further comprising:
providing at least one of the first reaction product and the second reaction product to a gas separator unit to recover a vapor fraction comprising compounds with a normal boiling point of 100 degrees C. or lower prior to providing the acid solution.

3. The method of claim 1 further comprising:
providing at least one of the acidified first reaction product and the acidified second reaction product to a gas separator unit to recover a vapor fraction comprising compounds with a normal boiling point of 100 degrees C. or lower.

4. The method 1 further comprising:
providing the aqueous stream to an aqueous separation zone to recover a light compounds from at least a portion of the aqueous phase in an overhead fraction, wherein the overhead fraction comprises a major portion of compounds with a normal boiling point of less than 150 degrees C. in said portion of the aqueous stream.

9. The method of claim 4 wherein the aqueous separation zone comprises at least one flasher to recover the overhead fraction.

10. The method of claim 9 further comprising:
providing to a further processing zone at least a portion of the vapor fraction, at least a portion of the overhead fraction from the aqueous stream, and/or at least a portion of the distillate overhead fraction to provide a higher hydrocarbon product.

11. The method of claim 1 further comprising:
providing an extraction solvent to the separation zone comprising a liquid-liquid extraction unit.

12. The method of claim 11 wherein the extraction solvent is at least a portion of the middle fraction from the distillation unit.

13. The method of claim 11 wherein the extraction solvent is at least a portion of the higher hydrocarbon product.

14. The method of claim 1 further comprising flashing at least a portion of the non-aqueous stream to recover an overhead fraction comprising light compounds and a bottom fraction comprising alkyl cyclohexanol, wherein the overhead fraction from the flashing comprises a major portion of compounds with a normal boiling point of less than 150 degrees C. in said portion of the non-aqueous stream, and wherein the bottom fraction from flashing comprises compounds with a normal boiling point of 150 degrees C. or higher;
providing a first portion of the bottom fraction from flashing to the liquid-liquid extraction unit; and
providing a second portion of the bottom fraction from flashing to the distillation unit.

15. The method of claim 1 further comprising:
at least partially depolymerizing lignin in the non-aqueous stream by providing at least a portion of the non-aqueous stream to a lignin depolymerization unit before providing it to the distillation unit.

16. The method of claim 15 further comprising:
providing at least a portion of the middle fraction from the distillation unit to the lignin depolymerization unit.

17. The method of claim 1 further comprising:
enhancing phase separation in the liquid-liquid phase separation unit by recycling at least a portion of the non-aqueous stream that exits the liquid-liquid phase separation unit with the first reaction product and/or the second reaction product entering to the liquid-liquid phase separation unit.

18. The method of claim 15 further comprising providing at least a portion of the higher hydrocarbon product to the at least partially depolymerized stream to precipitate at least a portion of the lignin in the stream to provide a slurry stream before providing it to the distillation unit.

19. A method comprising:
heating cellulosic biomass solids, molecular hydrogen, a catalyst capable of activating molecular hydrogen, and a digestion solvent in a reactor in a first reaction zone to produce a first reaction product;
heating at least a portion of the first reaction product, molecular hydrogen, and a catalyst capable of activating molecular hydrogen in a reactor in a second reaction zone to produce a second reaction product;
providing an acid solution to the second reaction product to produce an acidified second reaction product;
providing at least a portion of the acidified second reaction product to a separation zone comprising a liquid-liquid phase separation unit or a liquid-liquid extraction unit to recover an aqueous stream and a non-aqueous stream, wherein the aqueous stream comprises a major portion of water in said portion of the second reaction product;
providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and
providing at least a portion of the non-aqueous stream to a distillation unit to obtain at least an overhead fraction, a middle fraction, and a bottom fraction.

20. The method of claim 19 further comprising:
providing at least one of the first reaction product and the second reaction product to a gas separator unit to recover a vapor fraction comprising compounds with a normal boiling point of 100 degrees C. or lower prior to providing the acid solution.

21. The method of claim 20 further comprising:
providing at least one of the acidified first reaction product and the acidified second reaction product to a gas separator unit to recover a vapor fraction comprising compounds with a normal boiling point of 100 degrees C. or lower.

22. The method 19 further comprising:
providing the aqueous stream to an aqueous separation zone to recover a light compounds from at least a portion of the aqueous phase in an overhead fraction, wherein the overhead fraction comprises a major portion of compounds with a normal boiling point of less than 150 degrees C. in said portion of the aqueous stream.

23. The method of claim 22 wherein the aqueous separation zone comprises at least one flasher to recover the overhead fraction.

24. The method of claim 23 further comprising:
providing to a further processing zone at least a portion of the vapor fraction, at least a portion of the overhead fraction from the aqueous stream, and/or at least a portion of the distillate overhead fraction to provide a higher hydrocarbon product.

25. The method of claim 19 further comprising:
providing an extraction solvent to the separation zone comprising a liquid-liquid extraction unit.

26. The method of claim 25 wherein the extraction solvent is at least a portion of the middle fraction from the distillation unit.

27. The method of claim 25 wherein the extraction solvent is at least a portion of the higher hydrocarbon product.

28. The method of claim 19 further comprising:
at least partially depolymerizing lignin in the non-aqueous stream by providing at least a portion of the non-aqueous stream to a lignin depolymerization unit before providing it to the distillation unit.

29. A method comprising:
heating cellulosic biomass solids, molecular hydrogen, a catalyst capable of activating molecular hydrogen, and a digestion solvent in a reactor in a first reaction zone to produce a first reaction product;
providing at least a portion of the first reaction product to a separation zone comprising a liquid-liquid phase separation unit or a liquid-liquid extraction unit to recover an aqueous stream and a non-aqueous stream, wherein the aqueous stream comprises a major portion of water in said portion of the first reaction product,
providing an acid solution to the aqueous stream prior to providing the aqueous stream to an aqueous stream separation zone;
providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and
providing at least a portion of the non-aqueous stream to a distillation unit to obtain at least an overhead fraction, a middle fraction, and a bottom fraction.

30. The method of claim 29 further comprising:
providing at least one of the first reaction product and the second reaction product to a gas separator unit to recover a vapor fraction comprising compounds with a normal boiling point of 100 degrees C. or lower.

31. The method of 29 further comprising:
providing the aqueous stream to an aqueous separation zone to recover a light compounds from at least a portion of the aqueous phase in an overhead fraction, wherein the overhead fraction comprises a major portion of compounds with a normal boiling point of less than 150 degrees C. in said portion of the aqueous stream.

32. The method of claim 31 wherein the aqueous separation zone comprises at least one flasher to recover the overhead fraction.

33. The method of claim 32 further comprising:
providing to a further processing zone at least a portion of the vapor fraction, at least a portion of the overhead fraction from the aqueous stream, and/or at least a portion of the distillate overhead fraction to provide a higher hydrocarbon product.

34. The method of claim 29 further comprising:
providing an extraction solvent to the separation zone comprising a liquid-liquid extraction unit.

35. The method of claim 34 wherein the extraction solvent is at least a portion of the middle fraction from the distillation unit.

36. The method of claim 34 wherein the extraction solvent is at least a portion of the higher hydrocarbon product.

37. The method of claim 29 further comprising flashing at least a portion of the non-aqueous stream to recover an overhead fraction comprising light compounds and a bottom fraction comprising alkyl cyclohexanol, wherein the overhead fraction from the flashing comprises a major portion of compounds with a normal boiling point of less than 150 degrees C. in said portion of the non-aqueous stream, and wherein the bottom fraction from flashing comprises compounds with a normal boiling point of 150 degrees C. or higher;
providing a first portion of the bottom fraction from flashing to the liquid-liquid extraction unit; and
providing a second portion of the bottom fraction from flashing to the distillation unit.

38. The method of claim 29 further comprising:
at least partially depolymerizing lignin in the non-aqueous stream by providing at least a portion of the non-aqueous stream to a lignin depolymerization unit before providing it to the distillation unit.

39. The method of claim 38 further comprising:
providing at least a portion of the middle fraction from the distillation unit to the lignin depolymerization unit.

40. The method of claim 29 further comprising:
enhancing phase separation in the liquid-liquid phase separation unit by recycling at least a portion of the non-aqueous stream that exits the liquid-liquid phase separation unit with the first reaction product and/or the second reaction product entering to the liquid-liquid phase separation unit.

41. The method of claim 38 further comprising providing at least a portion of the higher hydrocarbon product to the at least partially depolymerized stream to precipitate at least a portion of the lignin in the stream to provide a slurry stream before providing it to the distillation unit.

42. The method of claim 31 wherein the aqueous separation zone comprises a first flasher and a second flasher, wherein the method further comprises:
providing the aqueous stream to the first flasher to recover a first overhead fraction and a first bottom fraction;
providing the first bottom fraction to the second flasher to recover middle-boiling compounds in a second overhead fraction and a second bottom fraction,
wherein the first overhead fraction comprises a major portion of compounds with a normal boiling point of less than 150 degrees C. in said portion of the aqueous stream, and
wherein the second overhead fraction comprises compounds with a normal boiling point of 150 degrees C. or higher.

43. The method of claim 41 wherein the slurry stream is provided to the distillation unit.

44. The method of claim 41 further comprising:
removing at least a portion of the precipitated lignin by filtration.

45. The method of claim 41 further comprising:
removing at least a portion of the precipitated lignin by hydrocyclone.

46. The method of claim 41 further comprising:
removing at least a portion of lignin in the non-aqueous stream prior to providing it to the distillation unit; and
providing at least a portion of the removed lignin to the lignin depolymerization unit.

47. The method of claim 29 wherein the acid solution is provided from a biomass acidic solution wash unit effluent.

48. The method of claim 29 wherein the acid solution comprises an inorganic acid.

49. The method of claim 48 wherein the inorganic acid is selected from the group consisting of sulfuric acid, nitric acid, phosphoric acid and hydrochloric acid.

50. The method of claim 1 further comprising:
providing at least a portion of the middle fraction to the reactor in the first reaction zone and/or the second reaction zone.

51. A method comprising:
heating cellulosic biomass solids, molecular hydrogen, a catalyst capable of activating molecular hydrogen, and a digestion solvent in a reactor in a first reaction zone to produce a first reaction product;
heating at least a portion of the first reaction product, molecular hydrogen, and a catalyst capable of activating molecular hydrogen in a reactor in a second reaction zone to produce a second reaction product;
providing at least a portion of the second reaction product to a separation zone comprising a liquid-liquid phase separation unit or a liquid-liquid extraction unit to recover an aqueous stream and a non-aqueous stream, wherein the aqueous stream comprises a major portion of water in said portion of the second reaction product,
providing an acid solution to the aqueous stream prior to providing the aqueous stream to an aqueous stream separation zone;
providing the aqueous stream to an aqueous stream separation zone to recover a light compounds from at least a portion of the aqueous phase in an overhead fraction, wherein the overhead fraction comprises a major portion of compounds with a normal boiling point of less than 150 degrees C. in said portion of the aqueous stream; and
providing at least a portion of the non-aqueous stream to a distillation unit to obtain at least an overhead fraction, a middle fraction, and a bottom fraction.

52. The method of claim 51 further comprising:
providing at least one of the first reaction product and the second reaction product to a gas separator unit to recover a vapor fraction comprising compounds with a normal boiling point of 100 degrees C. or lower.

53. The method of claim 51 further comprising:
providing the aqueous stream to an aqueous separation zone to recover a light compounds from at least a portion of the aqueous phase in an overhead fraction, wherein the overhead fraction comprises a major portion of compounds with a normal boiling point of less than 150 degrees C. in said portion of the aqueous stream.

54. The method of claim 53 wherein the aqueous separation zone comprises at least one flasher to recover the overhead fraction.

55. The method of claim 54 further comprising:
providing to a further processing zone at least a portion of the vapor fraction, at least a portion of the overhead fraction from the aqueous stream, and/or at least a portion of the distillate overhead fraction to provide a higher hydrocarbon product.

56. The method of claim 51 further comprising:
providing an extraction solvent to the separation zone comprising a liquid-liquid extraction unit.

57. The method of claim 56 wherein the extraction solvent is at least a portion of the middle fraction from the distillation unit.

58. The method of claim 56 wherein the extraction solvent is at least a portion of the higher hydrocarbon product.

59. The method of claim 51 further comprising flashing at least a portion of the non-aqueous stream to recover an overhead fraction comprising light compounds and a bottom fraction comprising alkyl cyclohexanol, wherein the overhead fraction from the flashing comprises a major portion of compounds with a normal boiling point of less than 150 degrees C. in said portion of the non-aqueous stream, and wherein the bottom fraction from flashing comprises compounds with a normal boiling point of 150 degrees C. or higher;
providing a first portion of the bottom fraction from flashing to the liquid-liquid extraction unit; and
providing a second portion of the bottom fraction from flashing to the distillation unit.

60. The method of claim 51 any preceding claim further comprising:
at least partially depolymerizing lignin in the non-aqueous stream by providing at least a portion of the non-aqueous stream to a lignin depolymerization unit before providing it to the distillation unit.

61. The method of claim 60 further comprising:
providing at least a portion of the middle fraction from the distillation unit to the lignin depolymerization unit.

62. The method of claim 51 further comprising:
enhancing phase separation in the liquid-liquid phase separation unit by recycling at least a portion of the non-aqueous stream that exits the liquid-liquid phase separation unit with the first reaction product and/or the second reaction product entering to the liquid-liquid phase separation unit.

63. The method of claim 60 further comprising providing at least a portion of the higher hydrocarbon product to the at least partially depolymerized stream to precipitate at least a portion of the lignin in the stream to provide a slurry stream before providing it to the distillation unit.

64. The method of claim 53 wherein the aqueous separation zone comprises a first flasher and a second flasher, wherein the method further comprises:
providing the aqueous stream to the first flasher to recover a first overhead fraction and a first bottom fraction;
providing the first bottom fraction to the second flasher to recover middle-boiling compounds in a second overhead fraction and a second bottom fraction,
wherein the first overhead fraction comprises a major portion of compounds with a normal boiling point of less than 150 degrees C. in said portion of the aqueous stream, and
wherein the second overhead fraction comprises compounds with a normal boiling point of 150 degrees C. or higher.

65. The method of claim 63 wherein the slurry stream is provided to the distillation unit.

66. The method of claim 63 further comprising:
removing at least a portion of the precipitated lignin by filtration.

67. The method of claim 63 further comprising:
removing at least a portion of the precipitated lignin by hydrocyclone.

68. The method of claim 63 further comprising:
removing at least a portion of lignin in the non-aqueous stream prior to providing it to the distillation unit; and
providing at least a portion of the removed lignin to the lignin depolymerization unit.

69. The method of claim 51 wherein the acid solution is provided from a biomass acidic solution wash unit effluent.

70. The method of claim 51 wherein the acid solution comprises an inorganic acid.

71. The method of claim 70 wherein the inorganic acid is selected from the group consisting of sulfuric acid, nitric acid, phosphoric acid and hydrochloric acid.

72. The method of claim 51 further comprising:
providing at least a portion of the middle fraction to the reactor in the first reaction zone and/or the second reaction zone.

73. A system comprising:
a first reaction zone comprising a reactor configured to heat cellulosic biomass solids, molecular hydrogen, a catalyst capable of activating molecular hydrogen, and a digestion solvent to form a first reaction product; and
a separation zone comprising:
an inlet to receive an acid solution in fluid communication with an outlet of the reactor in the first reaction zone and with the liquid-liquid phase separation unit or a liquid-liquid extraction unit;
a liquid-liquid phase separation unit or a liquid-liquid extraction unit having an inlet in fluid communication with an outlet of the reactor in the first reaction zone to receive the first reaction product, wherein the liquid-liquid phase separation unit or the liquid-liquid extraction unit is configured to provide an aqueous stream and a non-aqueous stream, wherein the aqueous phase comprises a major portion of water in said portion of the first reaction product;
an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and
a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction.

74. A system comprising:
a first reaction zone comprising a reactor configured heat cellulosic biomass solids, molecular hydrogen, a catalyst capable of activating molecular hydrogen, and a digestion solvent to form a first reaction product;
a second reaction zone comprising a reactor in fluid communication with an outlet of the reactor in the first reaction zone to receive the first reaction product, wherein the reactor in the second reaction zone is configured to heat a second reaction content comprising the first reaction product, molecular hydrogen, and a catalyst capable of activating molecular hydrogen to form a second reaction product; and
a separation zone comprising:
an inlet to receive an acid solution in fluid communication with an outlet of the reactor in the second reaction zone and with the liquid-liquid phase separation unit or the liquid-liquid extraction unit;
a liquid-liquid phase separation unit or a liquid-liquid extraction unit having an inlet in fluid communication with an outlet of the reactor in the second reaction zone to receive second first reaction product, wherein the liquid-liquid phase separation unit or the liquid-liquid extraction unit is configured to provide an aqueous stream and a non-aqueous stream, wherein the aqueous stream comprises a major portion of water in said portion of the first reaction product;
an aqueous stream separation zone to recover light compounds from at least a portion of the aqueous phase in an overhead fraction, wherein the overhead fraction comprises a major portion of compounds with a normal boiling point of less than 150 degrees C. in said portion of the aqueous stream, the aqueous stream separation zone having an inlet in fluid communication with an outlet of the liquid-liquid phase separation unit or a liquid-liquid extraction unit to receive the aqueous stream; and
a distillation unit having an inlet in fluid communication with an outlet of the liquid-liquid phase separation unit or the liquid-liquid extraction unit to receive at least a portion of the non-aqueous stream, wherein the distillation unit is configured to provide at least an overhead fraction, a middle fraction, and a bottom fraction;
wherein the distillation unit is in fluid communication with an inlet of the reactor in the first reaction zone to provide at least a portion of the middle fraction.
